(19) United States
(12) Patent Application Publication
(10) Pub. No.: US 2024/0002460 A1
(43) Pub. Date: Jan. 4, 2024

(54) A PROCESS FOR PRODUCING EGF

(71) Applicant: CHEMICAL & BIOPHARMACEUTICAL LABORATORIES OF PATRAS S.A., Patras (GR)

(72) Inventors: Kleomenis Barlos, Patras (GR); Konstantinos Barlos, Patras (GR)

(21) Appl. No.: 18/251,493

(22) PCT Filed: Nov. 5, 2021
(86) PCT No.: PCT/IB2021/060271

§ 371 Date: May 2, 2023

(30) Foreign Application Priority Data
Nov. 20, 2020 (GR) 20200100690

(51) Int. Cl. C07K 14/495

(52) U.S. Cl. CPC C07K 14/495

(57) ABSTRACT

The present invention relates to a process for preparing an epidermal growth factor-like peptide (EGF-like peptide) comprising the following amino acid sequence, C¹(X)₇C²(X)₄₋₅C³(X)₁₀₋₁₃C⁴(X)C⁵(X)₈C⁶ [SEQ NO: 49] wherein C¹-C⁶ are each cysteine and each X is independently a natural or unnatural amino acid, and wherein said EGF-like peptide has three intramolecular disulfide bonds; said process comprising the steps of: (I) preparing a first peptide fragment wherein the N-terminal amino acid is protected by a protecting group PG₁, which is selected from Boo and Fmoc; (II) preparing a second peptide fragment wherein the C-terminal amino acid is protected by a protecting group PG₂, which is selected from trityl, chlorotrityl and t-butyl; and wherein the amino acid side chains in said first and second peptide fragments are optionally protected; (III) coupling the C-terminal amino acid of said first peptide fragment with the N-terminal amino acid of said second peptide fragment in solution to form a linear protected EGF-like peptide; (IV)(a)(i) treating the linear protected EGF-like peptide formed in step (III) with iodine to form an oxidized mixture; (ii) globally deprotecting the oxidized mixture obtained in step (IV)(a)(i) by treating with trifluoroacetic acid (TFA); (iii) treating the deprotected oxidized mixture obtained in step (IV)(a)(ii) with DMSO/DTT to form a cmde EGF-like peptide; or (IV)(b)(i) globally deprotecting the linear protected EGF-like peptide obtained in step (III) by treating with trifluoroacetic acid (TFA); (ii) treating the deprotected mixture obtained in step (IV)(b)(i) with DMSO to form a cmde EGF-like peptide; and (V) optionally purifying the crude EGF-like peptide. Further aspects of the invention relate to processes for preparing EGF-like peptides using various fragment condensations.

### FIELD OF THE INVENTION

The present invention describes a process for the chemical synthesis of epidermal growth factor-like peptides (EGF-like peptides) and analogues and variants thereof, including, but not limited to, EGF and transforming growth factor-α (TGF-α).

### BACKGROUND OF THE INVENTION

Epidermal growth factor (EGF) is the founding member of the EGF-family of proteins. Members of this protein family have highly similar structural and functional characteristics. Besides EGF itself, other family members include Heparin-binding EGF-like growth factor (HB-EGF), transforming growth factor-α (TGF-α), Amphiregulin (AR), Epiregulin (EPR), Epigen, Betacellulin (BTC), neuregulin-1 (NRG1), neuregulin-2 (NRG2), neuregulin-3 (NRG3) and neuregulin-4 (NRG4).

All family members contain one or more repeats of the conserved amino acid sequence:

[SEQ ID NO: 49]
C¹(X)₇C²(X)₄₋₅C³(X)₁₀₋₁₃C⁴(X)C⁵(X)₈C⁶

wherein C¹-C⁶ are each cysteine and each X is independently an amino acid (Harris R. C., Chung E., Coffey, R. J., Experimental Cell Research 284 (2003) 2-13). The sequence contains six cysteine residues that form three intramolecular disulfide bonds (C¹-C³, C²-C⁴ and C⁵-C⁶). Disulfide bond formation generates three structural loops that are essential for high-affinity binding between members of the EGF-family and their cell-surface receptors.

EGF is a protein that stimulates cell growth and differentiation by binding to its receptor, EGFR. Human epidermal growth factor (hEGF) is a functionally versatile 6-kDa polypeptide comprising 53 amino acids and three intramolecular disulfide bonds, having the sequence:

[SEQ ID NO: 1]
H-Asn¹-Ser²-Asp³-Ser⁴-Glu⁵-Cys⁶-Pro⁷-Leu⁸-Ser⁹-His¹⁰-
Asp¹¹-Gly¹²-Tyr¹³-Cys¹⁴-Leu¹⁵-His¹⁶-Asp¹⁷-Gly¹⁸-Val¹⁹-
Cys²⁰-Met²¹-Tyr²²-Ile²³-Glu²⁴-Ala²⁵-Leu²⁶-Asp²⁷-Lys²⁸-
Tyr²⁹-Ala³⁰-Cys³¹-Asn³²-Cys³³-Val³⁴-Val³⁵-Gly³⁶-Tyr³⁷-
Ile³⁸-Gly³⁹-Glu⁴⁰-Arg⁴¹-Cys⁴²-Gln⁴³-Tyr⁴⁴-Arg⁴⁵-Asp⁴⁶-
Leu⁴⁷-Lys⁴⁸-Trp⁴⁹-Trp⁵⁰-Glu⁵¹-Leu⁵²-Arg⁵³-OH

The three intramolecular disulfide bonds are between Cys⁶ and Cys²⁰, Cys¹⁴ and Cys³¹, and Cys³³ and Cys⁴².

The Epidermal Growth Factor Receptor (EGFR) is one of the main targets of anticancer drugs and in anticancer drug development. Human epidermal growth factor (hEGF) is used in many of these therapeutic approaches as the vehicle to selectively transport the therapeutic active anticancer, antibody or anticancer agent to the hEGFR. In addition, several EGF-conjugates, such as fluorescently labeled EGF-like peptides, have been used as diagnostics. EGF like peptides, particularly hEGF, and human transforming growth factor (hTGF) have found several applications in cosmetic, skin care and medication, for example, in the treatment of diabetic foot ulcers (DFU).

One or more copies of EGF-like domains are contained in a large variety of functional proteins, including: Adipocyte differentiation inhibitor (gene PREF-1), Agrin, Amphiregulin, βcellulin, Blastula proteins BP10, BM86, Bone morphogenic protein 1 (BMP-1), Drosophila (the dorsal-ventral patterning protein tolloid), Caenorhabditis elegans developmental proteins lin-12 and glp-1, Caenorhabditis elegans apx-1 protein, Calcium-dependent serine proteinase (CASP), Cartilage matrix protein CMP, Cartilage oligomeric matrix protein COMP, Cell surface antigen 114/A10, Cell surface glycoprotein complex transmembrane subunit ASGP-2 Coagulation associated proteins C, Z and S, Coagulation factors VII, IX, X and XII, Complement C1r, Complement C1s, Complement-activating component of Ra-reactive factor (RARF), Complement components C6, C7, C8 α and β chains, and C9, Crumbs, Epidermal growth factor precursor, Exogastrula-inducing peptides A, C, D and X, Fat protein, Fetal antigen 1, Fibrillin 1 and fibrillin 2, Fibropellins IA, IB, IC, II and III, Fibulin-1 and -2, Giant-lens protein (protein Argos), Growth factor-related proteins from various poxviruses, Gurken protein, Heparin-binding EGF-like growth factor (HB-EGF), transforming growth factor α (TGF-α), growth factors Lin-3 and Spitz, Hepatocyte growth factor (HGF) activator. LDL and VLDL receptors, LDL receptor-related protein (LRP), Leucocyte antigen CD97, cell surface glycoprotein EMR1 and cell surface glycoprotein F4/80, Limulus clotting factor C, Meprin A a subunit, Milk fat globule-EGF factor 8 (MFG-E8), Neuregulin GGF-I and GGF-II, Neurexins, Neurogenic proteins Notch, Nidogen, Ookinete surface proteins (24 Kd, 25 Kd, 28 Kd), Pancreatic secretory granule membrane major glycoprotein GP2, Perforin, Proteoglycans aggrecan, versican, perlecan, brevican and chondroitin sulfate proteoglycan, Prostaglandin G/H synthase 1 and 2, Reelin, S1-5, Schwannoma-derived growth factor (SDGF), Selectins, Serine/threonine-protein kinase homolog (gene Pro25), Sperm-egg fusion proteins PH-30 α and β, Sperm flagellar membrane protein, Stromal cell derived protein-1 (SCP-1), TDGF-1, human teratocarcinoma-derived growth factor 1, Tenascin (or neuronectin), Thrombomodulin (fetomodulin), Thrombospondin 1, 2, 3 and 4, Thyroid peroxidase 1, Transforming growth factor β-1 binding protein (TGF-B1-BP), Tyrosine-protein kinase receptors Tek and Tie, Urokinase-type plasminogen activator and tissue plasminogen (TPA) Uromodulin, Vitamin K-dependent anticoagulants protein C and protein S and protein Z.

EGF-like peptides are generally produced by recombinant DNA technology. Chemical methods for preparing EGF-like peptides typically produce material in low yield and/or purity, are very laborious, and/or require the use of the very toxic and extremely corrosive HF.

The first total synthesis of urogastrone (h-EGF) was performed by the segment condensation of 10 small segments that were synthesized using Boc as the amino protecting group, Acm as the thiol protecting group, and Pac as the carboxy protecting group (Neya M., Hagiwara D., Miyazaki Y., Nakamura T., Hemmi K. and Hashimoto M., Journal of the Chemical Society, Perkin Transactions 1, 1989, Issue: 12, 2187-2198). The resulting linear protected EGF peptide was then deprotected with the toxic and extremely corrosive liquid HF. The use of HF is the cause of several side reactions, especially in the case of peptides containing sensitive nucleophilic amino acids such as Trp, Tyr, Met and Cys (hEGF contains in its sequence one Met, two Trp, five Tyr and six Cys residues). Besides the use of HF, this method is extremely laborious and is unsuitable for the large-scale production of pharmaceutical peptides.

In a similar way, the synthesis of EGF has also been performed by the fragment condensation of 9 protected fragments (Shin S. Y., Kaburaki Y., Watanabe M., and Munekata E., Biosci. Biotech. Biochem., 56 (3), 404-408, 1992; Neya M., Hagiwara D., Hemmi K., and Hashimoto M., J. Chem. Soc., Perkin Trans. 1, 1989, 2199-2205). These fragments were prepared in solution using Boc/Benzyl amino acids which were then condensed in solution sequentially. The resulting linear protected EGF peptide was then deprotected with liquid HF.

The synthesis of EGF in segments by solid phase peptide synthesis is described in the literature (Gell A. L., Groysbeck N., Becker C. F. W., Conibear A. C., J Pept Sci. 2017 Dec. 23 (12):871-879. doi: 10.1002/psc.3051. Epub 2017 Nov. 6). With a view to determine the structure of the EGF-like module of C1r and evaluate its contribution to calcium binding, C1r (123-175) was synthesized by automated solid-phase methodology using the Boc/Benzyl strategy. (Hernandez J. F., Bersch B., Pétillot Y., Arlaud G. J., J Pept Res. 1997, 49 (3), 221-31). Synthesis of the 40 amino acid epidermal growth factor-like domain of human cripto (also known as human teratocarcinoma-derived growth factor 1, TDGF-1) has also been described (Lohmeyer M., Harrison P. M., Kannan S., DeSantis M., O'Reilly N. J., Sternberg M. J. E., Salomon D. S., and Gullick W. J., Biochemistry, 1997, 36 (13), pp 3837-3845).

Transforming growth factor alpha (TGF-a) is another member of the EGF family. TGF-α is a protein that in humans is encoded by the TGFA gene. TGF-α is a ligand for the epidermal growth factor receptor, which activates a signaling pathway for cell proliferation, differentiation and development. The protein may act as either a transmembrane-bound ligand or a soluble ligand. TGF-α is upregulated in some human cancers. It is produced in macrophages, brain cells, and keratinocytes, and induces epithelial development.

TGF-α is synthesized internally as part of a 160 (human) or 159 (rat) amino acid transmembrane precursor (Ferrer I., Alcantara S., Ballabriga J., Olive M., Blanco R., Rivera R., Carmona M., Berruezo M., Pitarch S., Planas A.; Prog. Neurobiol. 1996, 49 (2), 99-123). The precursor is composed of an extracellular domain containing a hydrophobic transmembrane domain, 50 amino acids of TGF-α, and a 35-residue-long cytoplasmic domain. In its smallest form, TGF-α has six cysteines linked together via three disulfide bridges. Collectively, all members of the EGF/TGF-α family share this structure.

Step by step solid phase synthesis of the linear 50 amino acid residues of TGF-α was performed using Boc/Benzyl amino acids (Tam J. P., Sheikh M. A., Solomon D. S., and Ossowski L., Proc. Natl. Acad. Sci. USA, 83 (21), 8082-8086, 1986). The peptide was cleaved from the resin and deprotected with liquid HF. Again the use of HF renders this method unsuitable for scale up. Furthermore, the step-by-step synthesis of a lengthy peptide typically results in a mixture with a large number of very similar deletion and addition peptides that are extremely difficult to control and separate from the desired product. Accordingly, such methods are unsuitable for the large scale synthesis of pharmaceutical peptides.

To date, none of the existing methods for the bulk production of EGF-like peptides is completely satisfactory. The present invention therefore seeks to provide alternative methods for the synthesis of EGF-like peptides, ideally methods that are more efficient, and lead to improved yields and/or purity. In particular, there is a need to provide methods that are suitable for industrial scale-up, and which avoid the use of toxic or otherwise undesirable reagents.

### STATEMENT OF INVENTION

A first aspect of the invention relates to a process for preparing an epidermal growth factor-like peptide (EGF-like peptide) comprising the following amino acid sequence,

[SEQ ID NO: 49]
C¹(X)₇-C²(X)₄₋₅C³(X)₁₀₋₁₃C⁴(X)C⁵(X)₈C⁶

wherein C¹-C⁶ are each cysteine and each X is independently a natural or unnatural amino acid, and wherein said EGF-like peptide has three intramolecular disulfide bonds;

said process comprising the steps of:

(I) preparing a first peptide fragment wherein the N-terminal amino acid is protected by a protecting group PG₁, which is selected from Boc and Fmoc;

(II) preparing a second peptide fragment wherein the C-terminal amino acid is protected by a protecting group PG₂, which is selected from trityl, chlorotrityl and t-butyl;

and wherein the amino acid side chains in said first and second peptide fragments are optionally protected;

(III) coupling the C-terminal amino acid of said first peptide fragment with the N-terminal amino acid of said second peptide fragment in solution to form a linear protected EGF-like peptide;

(IV)(a)
(i) treating the linear protected EGF-like peptide formed in step (III) with iodine to form an oxidized mixture;
(ii) globally deprotecting the oxidized mixture obtained in step (IV)(a)(i) by treating with trifluoroacetic acid (TFA);
(iii) treating the deprotected oxidized mixture obtained in step (IV)(a)(ii) with DMSO/DTT to form a crude EGF-like peptide; or

(IV)(b)
(i) globally deprotecting the linear protected EGF-like peptide obtained in step (III) by treating with trifluoroacetic acid (TFA);
(ii) treating the deprotected mixture obtained in step (IV)(b)(i) with DMSO to form a crude EGF-like peptide; and

(V) optionally purifying the crude EGF-like peptide.

Advantageously, the process of the invention allows the chemical synthesis of human and murine EGF-like peptides by fragment condensation in excellent yield and purity. Morever, the process of the invention avoids the use of the extremely toxic and corrosive reagent HF, thereby rendering the process more suitable for the large scale manufacture of pharmaceutical peptides.

A second aspect of the invention relates to a process for preparing an EGF-like peptide comprising (or more preferably consisting of) the following sequence:

[SEQ ID NO: 1]
H-Asn¹-Ser²-Asp³-Ser⁴-Glu⁵-Cys⁶-Pro⁷-Leu⁸-Ser⁹-His¹⁰-
Asp¹¹-Gly¹²-Tyr¹³-Cys¹⁴-Leu¹⁵-His¹⁶-Asp¹⁷-Gly¹⁸-Val¹⁹-
Cys²⁰-Met²¹-Tyr²²-Ile²³-Glu²⁴-Ala²⁵-Leu²⁶-Asp²⁷-Lys²⁸-
Tyr²⁹-Ala³⁰-Cys³¹-Asn³²-Cys³³-Val³⁴-Val³⁵-Gly³⁶-Tyr³⁷-
Ile³⁸-Gly³⁹-Glu⁴⁰-Arg⁴¹-Cys⁴²-Gln⁴³-Tyr⁴⁴-Arg⁴⁵-Asp⁴⁶-
Leu⁴⁷-Lys⁴⁸-Trp⁴⁹-Trp⁵⁰-Glu⁵¹-Leu⁵²-Arg⁵³-OH

or a variant thereof,

wherein said process comprises:
coupling a first peptide fragment comprising (or more preferably consisting of) the sequence:
PG₁-Asn¹-Ser²-Asp³-Ser⁴-Glu⁵-Cys⁶-Pro⁷-Leu⁸-Ser⁹-His¹⁰-Asp¹¹-Gly¹²-Tyr¹³-Cys¹⁴-Leu¹⁵-His¹⁶-Asp¹⁷-Gly¹⁸-OH [SEQ ID NO: 2] or a variant thereof,
   wherein:
      PG₁ is an N-terminal protecting group selected from Boc and Fmoc; and
      the C-terminal amino acid is optionally in the form of an activated carboxylic acid derivative;
in solution with a second peptide fragment comprising (or more preferably consisting of) the sequence:
H-Val¹⁹-Cys²⁰-Met²¹-Tyr²²-Ile²³-Glu²⁴-Ala²⁵-Leu²⁶-AsP²⁷-Lys²⁸-Tyr²⁹-Ala³⁰-Cys³¹-Asn³²-Cys³³-Val³⁴-Val³⁵-Gly³⁶-Tyr³⁷-Ile³⁸-Gly³⁹-Glu⁴⁰-Arg⁴¹-Cys⁴²-Gln⁴³-Tyr⁴⁴-Arg⁴⁵-Asp⁴⁶-Leu⁴⁷-Lys⁴⁸-Trp⁴⁹-Trp⁵⁰-Glu⁵¹-Leu⁵²-Arg⁵³-O-PG₂ [SEQ ID NO: 3] or a variant thereof,
wherein PG₂ is a protecting group selected from chlorotrityl and t-butyl;
and wherein one or more of the amino acid residues in said first and second peptide fragments is optionally protected, preferably with an acid-cleavable protecting group; and
optionally removing protecting groups PG₁ and PG₂.

A third aspect of the invention relates to a process for preparing an EGF-like peptide comprising (or more preferably consisting of) the following sequence:

[SEQ ID NO: 1]
H-Asn¹-Ser²-Asp³-Ser⁴-Glu⁵-Cys⁶-Pro⁷-Leu⁸-Ser⁹-His¹⁰-
Asp¹¹-Gly¹²-Tyr¹³-Cys¹⁴-Leu¹⁵-His¹⁶-Asp¹⁷-Gly¹⁸-Val¹⁹-
Cys²⁰-Met²¹-Tyr²²-Ile²³-Glu²⁴-Ala²⁵-Leu²⁶-Asp²⁷-Lys²⁸-
Tyr²⁹-Ala³⁰-Cys³¹-Asn³²-Cys³³-Val³⁴-Val³⁵-Gly³⁶-Tyr³⁷-
Ile³⁸-Gly³⁹-Glu⁴⁰-Arg⁴¹-Cys⁴²-Gln⁴³-Tyr⁴⁴-Arg⁴⁵-Asp⁴⁶-
Leu⁴⁷-Lys⁴⁸-Trp⁴⁹-Trp⁵⁰-Glu⁵¹-Leu⁵²-Arg⁵³-OH

or a variant thereof,

and said process comprises:
coupling a first peptide fragment comprising (or more preferably consisting of) the sequence:
PG₁-Asn¹-Ser²-Asp³-Ser⁴-Glu⁵-Cys⁶-Pro⁷-Leu⁸-ψSer⁹-His¹⁰-Asp¹¹-Gly¹²-OH [SEQ ID NO: 4] or a variant thereof;
   wherein:
      PG₁ is an N-terminal protecting group selected from Boc and Fmoc; and
      the C-terminal amino acid is optionally in the form of an activated carboxylic acid derivative;
in solution with a second peptide fragment comprising (or more preferably consisting of) the sequence:
H-Tyr¹³-Cys¹⁴-Leu¹⁵-His¹⁶-Asp¹⁷-Gly¹⁸-Val¹⁹-Cys²⁰-Met²¹-Tyr²²-Ile²³-Glu²⁴-Ala²⁵-Leu²⁶-Asp²⁷-Lys²⁸-Tyr²⁹-Ala³⁰-Cys³¹-Asn³²-Cys³³-Val³⁴-Val³⁵-Gly³⁶-Tyr³⁷-Ile³⁸-Gly³⁹-Glu⁴⁰-Arg⁴¹-Cys⁴²-Gln⁴³-Tyr⁴⁴-Arg⁴⁵-Asp⁴⁶-Leu⁴⁷-Lys⁴⁸-Trp⁴⁹-Trp⁵⁰-Glu⁵¹-Leu⁵²-Arg⁵³-O-PG₂ [SEQ ID NO: 5] or a variant thereof;
wherein PG₂ is a protecting group selected from chlorotrityl and t-butyl;
and wherein one or more of the amino acid residues in said first and second peptide fragments is optionally protected, preferably with an acid-cleavable protecting group; and
optionally removing protecting groups PG₁ and PG₂.

A fourth aspect of the invention relates to a process for preparing an EGF-like peptide comprising (or more preferably consisting of) the following sequence:

H-Val¹-Val²-Ser³-His⁴-Phe⁵-Asn⁶-Asp⁷-Cys⁸-Pro⁹-Asp¹⁰-Ser¹¹-His¹²-Thr¹³-Gln¹⁴-Phe¹⁵-Cys¹⁶-Phe¹⁷-His¹⁸-Gly¹⁹-Thr²⁰-Cys²¹-Arg²²-Phe²³-Leu²⁴-Val²⁵-Gln²⁶-Glu²⁷-Asp²⁸-Lys²⁹-Pro³⁰-Ala³¹-Cys³²-Val³³-Cys³⁴-His³⁵-Ser³⁶-Gly³⁷-Tyr³⁸-Val³⁹-Gly⁴⁰-Ala⁴¹-Arg⁴²-Cys⁴³-Glu⁴⁴-His⁴⁵-Ala⁴⁶-Asp⁴⁷-Leu⁴⁸-Leu⁴⁹-Ala⁵⁰-OH [SEQ ID NO: 6] or a variant thereof;

and said process comprises:
coupling a first peptide fragment comprising (or more preferably consisting of) the sequence:
PG₁-Val¹-Val²-Ser³-His⁴-Phe⁵-Asn⁶-Asp⁷-Cys⁸-Pro⁹-Asp¹⁰-Ser¹¹-His¹²-Thr¹³-Gln¹⁴-Phe¹⁵-Cys¹⁶-Phe¹⁷-His¹⁸-Gly¹⁹-OH [SEQ ID NO: 7] or a variant thereof;
   wherein:
      PG₁ is an N-terminal protecting group selected from Boc and Fmoc; and
      the C-terminal amino acid is optionally in the form of an activated carboxylic acid derivative;
in solution with a second peptide fragment comprising (or more preferably consisting of) the sequence:
H-Thr²⁰-Cys²¹-Arg²²-Phe²³-Leu²⁴-Val²⁵-Gln²⁶-Glu²⁷-Asp²⁸-Lys²⁹-Pro³⁰-Ala³¹-Cys³²-Val³³-Cys³⁴-His³⁵-Ser³⁶-Gly³⁷-Tyr³⁸-Val³⁹-Gly⁴⁰-Ala⁴¹-Arg⁴²-Cys⁴³-Glu⁴⁴-His⁴⁵-Ala⁴⁶-Asp⁴⁷-Leu⁴⁸-Leu⁴⁹-Ala⁵⁰-O-PG₂ [SEQ ID NO: 8] or a variant thereof,
wherein PG₂ is a protecting group selected from chlorotrityl and t-butyl;
and wherein one or more of the amino acid residues in said first and second peptide fragments is optionally protected, preferably with an acid-cleavable protecting group; and
optionally removing protecting groups PG₁ and PG₂.

### DETAILED DESCRIPTION

### General Process for Preparing EGF-Like Peptides

A first aspect of the invention relates to a process as described above for preparing an epidermal growth factor-like peptide (EGF-like peptide) comprising the following amino acid sequence,

[SEQ ID NO: 49]
C¹(X)₇-C²(X)₄₋₅C³(X)₁₀₋₁₃C⁴(X)C⁵(X)₈C⁶

wherein C¹-C⁶ are each cysteine and each X is independently a natural or unnatural amino acid, and wherein said EGF-like peptide has three intramolecular disulfide bonds, between C¹ and C³, C² and C⁴, and C⁵ and C⁶.

In one preferred embodiment, the EGF-like peptide comprises from 1 to 20, or preferably from 2 to 15, or more preferably from 5 to 10 additional natural or unnatural amino acids at the N-terminus of [SEQ ID NO: 49]. In one particularly preferred embodiment, the EGF-like peptide comprises 5 additional natural amino acids at the N-terminus. In another particularly preferred embodiment, the EGF-like peptide comprises 7 additional natural amino acids at the N-terminus.

In one preferred embodiment, the EGF-like peptide comprises from 1 to 20, or preferably from 2 to 15, or more preferably from 5 to 12 or 5 to 10 additional natural or unnatural amino acids at the C-terminus of of [SEQ ID NO: 49]. In one particularly preferred embodiment, the EGF-like peptide comprises 11 additional natural amino acids at the C-terminus. In another particularly preferred embodiment, the EGF-like peptide comprises 7 additional natural amino acids at the C-terminus.

In one preferred embodiment, the EGF-like peptide comprises from 1 to 20, or preferably from 2 to 15, or more preferably from 5 to 10 additional natural or unnatural amino acids at the N-terminus of [SEQ ID NO: 49] and from 1 to 20, or preferably from 2 to 15, or more preferably from 54 to 12 or 5 to 10 additional natural or unnatural amino acids at the C-terminus of of [SEQ ID NO: 49]. In one particularly preferred embodiment, the EGF-like peptide comprises 5 additional natural amino acids at the N-terminus and 11 additional natural amino acids at the C-terminus. In another particularly preferred embodiment, the EGF-like peptide comprises 7 additional natural amino acids at the N-terminus and additional natural amino acids at the C-terminus.

As used herein, the term “non-natural amino acid” or “unnatural amino acid” includes alpha and alpha-disubstituted amino acids, N-alkyl amino acids, lactic acid, halide derivatives of natural amino acids such as trifluorotyrosine, p-Cl-phenylalanine, p-F-phenylalanine, p-Br-phenylalanine, p-NO₂-phenylalanine, phenylglycine, sarcosine, penicillamine, D-2-methyltryptophan, phosphoserine, phosphothreonine, phosphotyrosine, p-I-phenylalanine, L-allyl-glycine, β-alanine, β-aspartic acid, β-cyclohexylalanine, citrulline, homoserine, homocysteine, pyroglutamic acid, L-α-amino butyric acid, L-γ-amino butyric acid, L-α-amino isobutyric acid, α-cyclohexylglycine, diaminobutyric acid, diaminopimelic acid, N-ϵ-dinitrophenyl-lysine, L-1-naphthylalanine, L-2-naphthylalanine, 3-(2-pyridyl)-L-alanine, 3-(3-pyridyl)-L-alanine, 3-(4-pyridyl)-L-alanine, N-ϵ-methyl-lysine, N,N-ϵ-dimethyl-lysine, N,N,N-ϵ-trimethyl-lysine, 3-mercaptopropionic acid, L-ϵ-amino caproic acid, 7-amino heptanoic acid, 6-amino hexanoic acid L-methionine sulfone, ornithine, L-norleucine, L-norvaline, p-nitro-L-phenylalanine, L-hydroxyproline, γ-glutamic acid, γ-amino butyric acid L-thioproline, methyl derivatives of phenylalanine (Phe) such as 4-methyl-Phe, pentamethyl-Phe, L-Phe (4-amino), L-Tyr (methyl), L-Phe (4-isopropyl), L-Tic (1,2,3,4tetrahydroiso-quinoline-3-carboxyl acid), L-diaminopropionic acid and L-Phe (4-benzyl).

In one preferred embodiment, each X is independently a natural amino acid selected from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

In one preferred embodiment, the EGF-like protein is selected from: EGF, Heparin-binding EGF-like growth factor (HB-EGF), transforming growth factor-α (TGF-α), Amphiregulin (AR), Epiregulin (EPR), Epigen, Betacellulin (BTC), neuregulin-1 (NRG1), neuregulin-2 (NRG2), neuregulin-3 (NRG3) and neuregulin-4 (NRG4). More preferably, the EGF-like protein is selected from EGF and transforming growth factor-α (TGF-α).

In one particularly preferred embodiment, the EGF-like protein is EGF, more preferably human or murine EGF, even more preferably, human EGF.

In another particularly preferred embodiment, the EGF-like protein is transforming growth factor-α (TGF-α), more preferably human TGF-α.

In addition to the specific peptides mentioned herein, the invention also encompasses variants, derivatives, analogues, homologues and fragments thereof.

As used herein, a “variant” of any given sequence is a sequence in which the specific sequence of amino acid residues has been modified in such a manner that the peptide in question retains at least one of its endogenous functions. A variant sequence can be obtained by addition, deletion, substitution, modification, replacement and/or variation of at least one residue present in the naturally occurring peptide.

The term “derivative” as used herein in relation to peptides described herein includes any substitution of, variation of, modification of, replacement of, deletion of and/or addition of one (or more) amino acid residues from or to the sequence, providing that the resultant peptide retains at least one of its endogenous functions.

The term “analogue” as used herein in relation to peptides includes any mimetic, that is, a chemical compound that possesses at least one of the endogenous functions of the peptides which it mimics.

Typically, amino acid substitutions may be made, for example from 1, 2 or 3, to 10 or substitutions, provided that the modified sequence retains the required activity or ability. Amino acid substitutions may include the use of non-naturally occurring analogues.

Peptides described herein may also have deletions, insertions or substitutions of amino acid residues which produce a silent change and result in a functionally equivalent peptide. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence. The term “homology” can be equated with “identity”.

In the present context, a homologous sequence is taken to include an amino acid sequence which may be at least 50%, 55%, 65%, 75%, 85% or 90% identical, preferably at least 95%, 96% or 97% or 98% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye, or more usually, with the aid of readily available sequence comparison programs. These commercially available computer programs can calculate percent homology or identity between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid sequence may cause the following residues or codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, USA; Devereux et al. (1984) Nucleic Acids Research 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, BLAST 2 Sequences, is also available for comparing protein and nucleotide sequences (FEMS Microbiol. Lett. (1999) 174(2):247-50; FEMS Microbiol. Lett. (1999) 177(1):187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the peptide that is of interest either functionally or, for example, in an assay. “Fragment” thus refers to an amino acid sequence that is a portion of a full-length peptide.

More preferably, the term “variant” includes any variation wherein wherein (a) one or more amino acid residues are replaced by a naturally or non-naturally occurring amino acid residue (b) the order of two or more amino acid residues is reversed, (c) one, two or three amino acids are deleted, (d) a spacer group is present between any two amino acid residues, (e) one or more amino acid residues are in peptoid form, (f) the (N-C-C) backbone of one or more amino acid residues of the peptide has been modified, (g) one or more additional amino acids are present at the N-terminus and/or the C-terminus, or any of (a)-(g) in combination. Preferably, the variants arise from one of (a), (b) or (c).

The present invention also encompasses amino acid sequences modified by the incorporation of one or more pseudoprolines (denoted ψ). Pseudoprolines are artificially created dipeptides that minimize aggregation during FMOC solid phase synthesis of peptides. Pseudoprolines consist of serine-(Oxa) or threonine-derived oxazolidines [Oxa(5-Me)] and Cysteine-derived thiazolidines (THz) with Proline-like ring structures (see below).

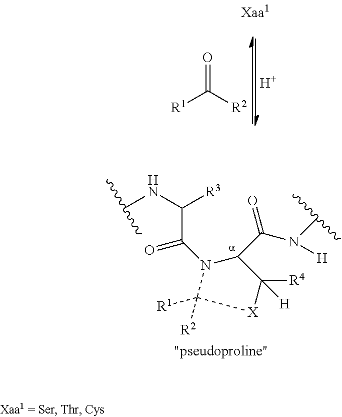

Due to the preference for a cis-amide bond with the preceding residue of C2-substituted pseudoprolines, their incorporation results in a kink conformation of the peptide backbone, thereby preventing peptide aggregation, self-association, or β-structure formation. Hence, pseudoprolines fulfil two functions simultaneously: firstly, they serve as temporary side-chain protection for Ser, Thr, and Cys, and secondly they act as solubilizing building blocks to increase solvation and coupling rates during peptide synthesis and in subsequent chain assembly.

Pseudoprolines are obtained by reacting the free amino acids with aldehydes or ketones. Pseudoproline dipeptides can be introduced in the same manner as other amino acid derivatives. Preferably the pseudoproline is derived from a Ser-X, Thr-X or Cys-X group, where X is a natural or unnatural amino acid. The routine use of pseudoproline (oxazolidine) dipeptides in the FMOC solid phase peptide synthesis (SPPS) of serine- and threonine-containing peptides leads to significant improvements in quality and yield of crude products. Once the peptide is deprotected, the pseuoproline becomes a conventional dipeptide of the form X-Ser, X-Thr or X-Cys, wherein X is a natural or unnatural amino acid.

More preferably, the variant has one to five, or one to four, or one to three amino acids residues substituted by one or more other amino acid residues. Even more preferably, two amino acid residues are substituted by another amino acid residue. More preferably still, one amino acid residue is substituted by another amino acid residue. Preferably, the substitution is homologous.

Homologous substitution (substitution and replacement are both used herein to mean the interchange of an existing amino acid residue, with an alternative residue) may occur, i.e. like-for-like substitution such as basic for basic, acidic for acidic, polar for polar etc. Non-homologous substitution may also occur i.e. from one class of residue to another or alternatively involving the inclusion of unnatural amino acids such as ornithine, diaminobutyric acid ornithine, norleucine ornithine, pyridylalanine, thienylalanine, naphthylalanine and phenylglycine, a more detailed list of which appears below. More than one amino acid residue may be modified at a time.

Suitable spacer groups that may be inserted between any two amino acid residues of the carrier moiety include alkyl groups such as methyl, ethyl or propyl groups in addition to amino acid spacers such as glycine or β-alanine residues. A further form of variation, type (e), involving the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example, Simon R J et al., PNAS (1992) 89 (20), 9367-9371 and Horwell D C, Trends Biotechnol. (1995) 13 (4), 132-134. Type (f) modification may occur by methods such as those described in International Application PCT/GB99/01855 (WO 99/64574).

It is preferable for amino acid variation, preferably of type (a) or (b), to occur independently at any position. As mentioned above more than one homologous or non-homologous substitution may occur simultaneously. Further variation may occur by virtue of reversing the sequence of a number of amino acid residues within a sequence.

In one embodiment the replacement amino acid residue is a natural amino acid selected from the residues of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. The replacement amino acid residue may additionally be selected from unnatural amino acids.

The process of the invention comprises the steps of:

(I) preparing a first peptide fragment wherein the N-terminal amino acid is protected by a protecting group PG₁, which is selected from Boc and Fmoc;

(II) preparing a second peptide fragment wherein the C-terminal amino acid is protected by a protecting group PG₂, which is selected from trityl, chlorotrityl and t-butyl;

and wherein the amino acid side chains in said first and second peptide fragments are optionally protected;

(III) coupling the C-terminal amino acid of said first peptide fragment with the N-terminal amino acid of said second peptide fragment in solution to form a linear protected EGF-like peptide;

(IV)(a)
(i) treating the linear protected EGF-like peptide formed in step (III) with iodine to form an oxidized mixture;
(ii) globally deprotecting the oxidized mixture obtained in step (IV)(a)(i) by treating with trifluoroacetic acid (TFA);
(iii) treating the deprotected oxidized mixture obtained in step (IV)(a)(ii) with DMSO/DTT to form a crude EGF-like peptide; or

(IV)(b)
(i) globally deprotecting the linear protected EGF-like peptide obtained in step (III) by treating with trifluoroacetic acid (TFA);
(ii) treating the deprotected mixture obtained in step (IV)(b)(i) with DMSO to form a crude EGF-like peptide; and

(V) optionally purifying the crude EGF-like peptide.

Step (I) of the process comprises preparing a first peptide fragment wherein the N-terminal amino acid is protected by a protecting group PG₁, which is selected from Boc and Fmoc.

In one preferred embodiment, the first peptide fragment is prepared by coupling two or more peptide sub-fragments.

In another preferred embodiment, the first peptide fragment is prepared by solid phase peptide synthesis.

Step (II) of the process comprises preparing a second peptide fragment wherein the C-terminal amino acid is protected by a protecting group PG₂, which is selected from chlorotrityl and t-butyl.

In one preferred embodiment, the second peptide fragment is prepared by coupling two or more peptide sub-fragments.

In one preferred embodiment, the second peptide fragment is prepared by solid phase peptide synthesis.

Step (III) of the process comprises coupling the C-terminal amino acid of said first peptide fragment with the N-terminal amino acid of said second peptide fragment in solution to form a protected EGF-like peptide. The respective sequences of the first and second peptide fragments are such that their coupling in step (III) gives rise to a peptide of the sequence C¹(X)₇C²(X)₄₋₅C³(X)₁₀₋₁₃C⁴(X)C⁵(X)₈C⁶ [SEQ ID NO: 49] which is in protected, linear form. This linear, protected peptide is subsequently deprotected, and undergoes rearrangement to form the final EGF-like peptide having the correct arrangement of intramolecular disulfide bonds (see Step (IV)(a) or (IV)(b)). For example, where the EGF-like peptide is EGF (see below), three intramolecular disulfide bonds are formed between Cys⁶ and Cys²⁰, Cys¹⁴ and Cys³¹, and Cys³³ and Cys⁴².

Preferably, the first peptide fragment is activated, for example, by treating with HOBt. H₂O, and then coupled with the second peptide fragment in the presence of a coupling reagent and a solvent. Preferably, acid activation (for example, with HOBt. H₂O) applies to all fragment condensation reactions described herein where a first peptide fragment terminating in a COOH group is coupled with the free NH₂ group of a second peptide fragment.

HOBt is used to produce an activated ester. The resulting ester then reacts with the amine group of the second peptide fragment to form an amide bond. Other benzotriazole activating agents may also be used and would be familiar to the skilled person. Suitable alternatives, include, but are not limited to chloro benzotriazole and aza benzotriazole. Activation and coupling can also be performed using uranium salts such as HBTU, TBTU and the like.

Suitable coupling reagents will be familiar to the skilled person and include, for example, carbodiimide coupling reagents such as DIC (N,N′-diisopropylcarbodiimide), DCC (N,N′-Dicyclohexylcarbodiimide) and EDAC (1-ethyl-3-(3-dimethylaminopropyl) carbodiimide). Preferably, the coupling reagent is EDAC, more preferably in the form of its HCl salt.

Suitable solvents for the coupling step will be familiar to the skilled person. Preferably, the solvent is selected from N-methyl pyrrolidone (NMP), dimethyl formamide (DMF), dimethyl acetamide (DMAC), dichloromethane (DCM) and mixtures thereof. More preferably, the solvent is NMP.

The process of the invention then proceeds either via Step (IV)(a) or Step (IV)(b) as described below.

In one preferred embodiment, the process proceeds via Step (IV)(a). Step (IV)(a) comprises steps (i)-(iii). Step (IV)(a)(i) comprises treating the protected EGF-like peptide formed in step (III) with iodine to form an oxidized mixture. The iodine simultaneously removes the cysteine side-chain protecting groups, and then oxidises the cysteines to form disulfides. This results in a wide range of different products in which the cysteine residues are cross-linked with one other. Preferably, the iodine oxidation step (IV)(a)(i) takes place in a suitable organic solvent. Suitable solvents will be familiar to the skilled person and include, for example, dichloromethane. Preferably, the iodine oxidation step is carried out using a solution of iodine in TFA/dichloromethane, more preferably a 1% solution of iodine in TFA/dichloromethane.

Step (IV)(a)(ii) of the process comprises globally deprotecting the oxidized mixture obtained in step (IV)(a)(i) by treating with trifluoroacetic acid (TFA). This step removes all of the remaining protecting groups from the peptide. Preferably, this step comprises treating the oxidized mixture obtained in step (IV)(a)(i) with a mixture comprising TFA/H₂O/DTT, more preferably in a ratio of 94:3:3. The DTT functions as a scavenger and to avoid possible premature oxidation.

Step (IV)(a)(iii) of the process comprises treating the deprotected oxidized mixture obtained in step (IV)(a)(ii) with dithiothreitol (DTT) and DMSO to form a crude EGF-like peptide. DTT is a reducing agent for disulfide bonds, and DMSO is a mild oxidant. Treating with DMSO and DTT causes the disulfide bonds to equilibrate (or “reshuffle”) so as to form the most thermodynamically favourable product. This process is known as oxidative folding and yields an EGF-like peptide having the correct disulfide bond configuration to form the loop structures that are essential for recognition by EGFR. Preferably, this step takes place in water/DMSO. More preferably, this step takes place in a DMSO and water solution comprising Tris and guanidine hydrochloride, where the latter functions as a chaotrope. In another embodiment, step (IV)(a)(iii) of the process comprises treating the deprotected oxidized mixture obtained in step (IV)(a)(ii) with a DMSO and water solution comprising Tris and guanidine hydrochloride to form a crude EGF-like peptide.

In an alternative preferred embodiment, the process proceeds via Step (IV)(b). Step (IV)(b) comprises steps (i)-(ii). Step (IV)(b)(i) comprises globally deprotecting the linear protected EGF-like peptide obtained in step (III) by treating with trifluoroacetic acid (TFA). This step removes all of the remaining protecting groups from the peptide. Preferably, this step comprises treating the oxidized mixture obtained in step (III) with a mixture comprising TFA/H₂O/DTT, more preferably in a ratio of 94:3:3. The DTT functions as a scavenger and to avoid possible premature oxidation.

Step (IV)(b)(ii) comprises treating the deprotected mixture obtained in step (IV)(b)(i) with DMSO to form a crude EGF-like peptide. In a preferred embodiment, step (IV)(b)(ii) of the process comprises treating the deprotected oxidized mixture obtained in step (IV)(b)(i) with a DMSO and water solution comprising Tris and guanidine hydrochloride to form a crude EGF-like peptide.

Step (V) of the process comprises optionally purifying the crude EGF-like peptide. Suitable purification methods are commonly known in the art and include, for example, HPLC. The skilled person will be familiar with suitable solvents and column materials for the HPLC purification of peptides. Suitable solvents, column materials and conditions for purification are exemplified in the accompanying Examples.

In one preferred embodiment, the first peptide fragment is prepared by coupling two or more peptide sub-fragments.

In one preferred embodiment, the first peptide fragment is prepared by solid phase peptide synthesis.

In one preferred embodiment, the second peptide fragment is prepared by coupling two or more peptide sub-fragments.

In one preferred embodiment, the second peptide fragment is prepared by solid phase peptide synthesis.

### Process for Preparing Epidermal Growth Factor (EGF)

In one preferred embodiment, the EGF-like peptide is EGF, or an analogue or variant thereof.

In one preferred embodiment, the EGF-like peptide is murine EGF, or an analogue or variant thereof.

In one preferred embodiment, the EGF-like peptide is human EGF, or an analogue or variant thereof.

In one preferred embodiment, the C-terminal amino acid of the first peptide fragment is glycine.

In one preferred embodiment, the EGF-like peptide comprises (or more preferably consists of) the following sequence:

[SEQ ID NO: 1]
H-Asn¹-Ser²-Asp³-Ser⁴-Glu⁵-Cys⁶-Pro⁷-Leu⁸-Ser⁹-His¹⁰-
Asp¹¹-Gly¹²-Tyr¹³-Cys¹⁴-Leu¹⁵-His¹⁶-Asp¹⁷-Gly¹⁸-Val¹⁹-
Cys²⁰-Met²¹-Tyr²²-Ile²³-Glu²⁴-Ala²⁵-Leu²⁶-Asp²⁷-Lys²⁸-
Tyr²⁹-Ala³⁰-Cys³¹-Asn³²-Cys³³-Val³⁴-Val³⁵-Gly³⁶-Tyr³⁷-
Ile³⁸-Gly³⁹-Glu⁴⁰-Arg⁴¹-Cys⁴²-Gln⁴³-Tyr⁴⁴-Arg⁴⁵-Asp⁴⁶-
Leu⁴⁷-Lys⁴⁸-Trp⁴⁹-Trp⁵⁰-Glu⁵¹-Leu⁵²-Arg⁵³-OH

or a variant thereof,

and said process comprises:
coupling a first peptide fragment comprising (or more preferably consisting of) the sequence:
PG₁-Asn¹-Ser²-Asp³-Ser⁴-Glu⁵-Cys⁶-Pro⁷-Leu⁸-Ser⁹-His¹⁰-Asp¹¹-Gly¹²-Tyr¹³-Cys¹⁴-Leu¹⁵-His¹⁶-Asp¹⁷-Gly¹⁸-OH [SEQ ID NO: 2] or a variant thereof,
   wherein:
      PG₁ is an N-terminal protecting group selected from Boc and Fmoc; and
      the C-terminal amino acid is optionally in the form of an activated carboxylic acid derivative;
in solution with a second peptide fragment comprising (or more preferably consisting of) the sequence:
H-Val¹⁹-Cys²⁰-Met²¹-Tyr²²-Ile²³-Glu²⁴-Ala²⁵-Leu²⁶-Asp²⁷-Lys²⁸-Tyr²⁹-Ala³⁰-Cys³¹-Asn³²-Cys³³-Val³⁴-Val³⁵-Gly³⁶-Tyr³⁷-Ile³⁸-Gly³⁹-Glu⁴⁰-Arg⁴¹-Cys⁴²-Gln⁴³-Tyr⁴⁴-Arg⁴⁵-Asp⁴⁶-Leu⁴⁷-Lys⁴⁸-Trp⁴⁹-Trp⁵⁰-Glu⁵¹-Leu⁵²-Arg⁵³-O-PG₂ [SEQ ID NO: 3] or a variant thereof,
wherein PG₂ is a protecting group selected from chlorotrityl and t-butyl;
and wherein one or more of the amino acid residues in said first and second peptide fragments is optionally protected, preferably with an acid-cleavable protecting group.

Acid cleavable protecting groups include, but are not limited to, ^{t}Bu, Boc, Acm, O^{t}Bu, Trt, Mmt, Mtt and Pbf. In one preferred embodiment, one or more amino acid side chains the amino acid residues in said first and second peptide fragments is optionally protected protected with an acid-cleavable protecting group selected from ^{t}Bu, Ttr, Pbf and Boc.

In one preferred embodiment, the EGF-like peptide comprises (or more preferably consists of) the following sequence:

H-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-Tyr¹³(P)-Cys¹⁴(P)-Leu¹⁵-His¹⁶(P)-Asp¹⁷(P)-Gly¹⁸-Val¹⁹-Cys²⁰(P)-Met²¹-Tyr²²(P)-Ile²³-Glu²⁴(P)-Ala²⁵-Leu²⁶-Asp²⁷(P)-Lys28(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-OH [SEQ ID NO: 9] or a variant thereof,

wherein the first peptide fragment comprises (or more preferably consists of) the following sequence:

PG₁-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-Tyr¹³(P)-Cys¹⁴(P)-Leu¹⁵-His¹⁶(P)-Asp¹⁷(P)-Gly¹⁸-OH [SEQ ID NO: 10] or a variant thereof;

and the second peptide fragment comprises (or more preferably consists of) the following sequence:

H-Val¹⁹-Cys²⁰P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 11] or a variant thereof;

wherein each P represents a side chain protecting group which may be the same or different.

In one preferred embodiment, the EGF-like peptide comprises (or more preferably consists of) the following sequence:

H-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-Tyr¹³(tBu)-Cys¹⁴(Trt)-Leu¹⁵-His¹⁶(Trt)-Asp¹⁷(tBu)-Gly¹⁸-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-OH [SEQ ID NO: 12] or a variant thereof;

and wherein the first peptide fragment comprises (or more preferably consists of) the following sequence:

PG₁-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-Tyr¹³(tBu)-Cys¹⁴(Trt)-Leu¹⁵-His¹⁶(Trt)-Asp¹⁷(tBu)-Gly¹⁸-OH [SEQ ID NO: 13] or a variant thereof,

and the second peptide fragment comprises (or more preferably consists of) the following sequence:

[SEQ ID NO: 14]
H-Val¹⁹-Cys²⁰(Trt)-Met²¹-Tyr²²(tBu)-Ile²³-Glu²⁴(tBu)-
Ala²⁵-Leu²⁶-Asp²⁷(tBu)-Lys²⁸(Boc)-Tyr²⁹(tBu)-Ala³⁰-
Cys³¹(Trt)-Asn³²(Trt)-Cys³³(Trt)-Val³⁴-Val³⁵-Gly³⁶-
Tyr³⁷(tBu)-Ile³⁸-Gly³⁹-Glu(tBu)⁴⁰-Arg⁴¹(Pbf)-Cys⁴²
(Trt)-Gln⁴³(Trt)-Tyr⁴⁴(tBu)-Arg⁴⁵(Pbf)-Asp⁴⁶(tBu)-
Leu⁴⁷-Lys⁴⁸(Boc)-Trp⁴⁹(Boc)-Trp⁵⁰(Boc)-Glu⁵¹(tBu)-
Leu⁵²-Arg⁵³(Pbf)-O-PG₂

or a variant thereof.

In one preferred embodiment, the first peptide fragment PG₁-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-Tyr¹³(P)-Cys¹⁴(P)-Leu¹⁵-His¹⁶(P)-Asp¹⁷(P)-Gly¹⁸-OH [SEQ ID NO: 10] or PG₁-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-Tyr¹³(tBu)-Cys¹⁴(Trt)-Leu¹⁵-His¹⁶(Trt)-Asp¹⁷(tBu)-Gly¹⁸-OH [SEQ ID NO: 13] is prepared by solid phase synthesis starting from Fmoc-Gly-OH.

In one preferred embodiment, the first peptide fragment PG₁-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-Tyr¹³(P)-Cys¹⁴(P)-Leu¹⁸-His¹⁸(P)-Asp¹⁷(P)-Gly¹⁸-OH [SEQ ID NO: 15] is prepared by fragment condensation of PG₁-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-OH [SEQ ID NO: 16] and H-Tyr¹³(P)-Cys¹⁴(P)-Leu¹⁵-His¹⁶(P)-Asp¹⁷(P)-Gly¹⁸-O-PG₂ [SEQ ID NO: 17]. Preferably, the protecting group PG₂ is then removed.

In one preferred embodiment, the first peptide fragment PG₁-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-Tyr¹³(tBu)-Cys¹⁴(Trt)-Leu¹⁵-His¹⁶(Trt)-Asp¹⁷(tBu)-Gly¹⁸-OH [SEQ ID NO: 13] is prepared by fragment condensation of PG₁-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-OH [SEQ ID NO: 18] and H-Tyr¹³(tBu)-Cys¹⁴(Trt)-Leu¹⁵-His¹⁶(Trt)-Asp¹⁷(tBu)-Gly¹⁸-O-PG₂[SEQ ID NO: 19]. Preferably, the protecting group PG₂ is then removed.

In one preferred embodiment, the peptide fragment PG₁-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-OH [SEQ ID NO: 20] or PG₁-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-OH [SEQ ID NO: 21] is prepared by solid phase synthesis starting from Fmoc-Gly-OH.

In one preferred embodiment, the peptide fragment H-Tyr¹³(P)-Cys¹⁴(P)-Leu¹⁵-His¹⁶(P)-Asp¹⁷(P)-Gly¹⁸-O-PG₂ [SEQ ID NO: 17] or H-Tyr¹³(tBu)-Cys¹⁴(Trt)-Leu¹⁵-His¹⁶(Trt)-Asp¹⁷(tBu)-Gly¹⁸-O-PG₂ [SEQ ID NO: 19] is prepared by solid phase synthesis starting from Fmoc-Gly-OH.

In one preferred embodiment, the second peptide fragment H-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 11] is prepared by fragment condensation of PG₁-Val¹⁹-Cys²⁰P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-OH [SEQ ID NO: 22] and H-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 23]. Preferably, the protecting group PG₁ is then removed.

In one preferred embodiment, the second peptide fragment H-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 14] is prepared by fragment condensation of PG₁-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-OH [SEQ ID NO: 24] and H-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 25]. Preferably, the protecting group PG₁ is then removed.

In one preferred embodiment, the second peptide fragment H-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 11] is prepared by fragment condensation of PG₁-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-OH [SEQ ID NO: 26] and H-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 27]. Preferably, the protecting group PG₁ is then removed.

In one preferred embodiment, the second peptide fragment H-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 14] is prepared by fragment condensation of PG₁-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-OH [SEQ ID NO: 28] and H-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 29]. Preferably, the protecting group PG₁ is then removed.

In one preferred embodiment, the peptide fragment H-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 23] is prepared by fragment condensation of PG₁-Tyr³⁷(P)-Ile³⁸-Gly³⁹-OH [SEQ ID NO: 30] and H-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 27]. Preferably, the protecting group PG₁ is then removed.

In one preferred embodiment, the peptide fragment H-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 25] is prepared by fragment condensation of PG₁-Tyr³⁷(tBu)-Ile³⁸-Gly³⁹-OH [SEQ ID NO: 31] and H-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 29]. Preferably, the protecting group PG₁ is then removed.

In one preferred embodiment, the peptide fragment PG₁-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-OH [SEQ ID NO: 22] or PG₁-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-OH [SEQ ID NO: 24] is prepared by solid phase peptide synthesis starting with Fmoc-Gly-OH.

In one preferred embodiment, the peptide fragment H-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 23] or H-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁶³(Pbf)-O-PG₂ [SEQ ID NO: 25] is prepared by solid phase peptide synthesis starting with Fmoc-Arg(P) or Fmoc-Arg(Pbf).

In one preferred embodiment, the peptide fragment PG₁-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-OH [SEQ ID NO: 26] or PG₁-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-OH [SEQ ID NO: 28] is prepared by solid phase peptide synthesis starting with Fmoc-Gly-OH.

In one preferred embodiment, the peptide fragment H-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 27] or H-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 29] is prepared by solid phase peptide synthesis using Fmoc-Arg(P) or Fmoc-Arg(Pbf).

In one preferred embodiment, the EGF-like peptide comprises (or more preferably consists of) the following sequence:

[SEQ ID NO: 1]
H-Asn¹-Ser²-Asp³-Ser⁴-Glu⁵-Cys⁶-Pro⁷-Leu⁸-Ser⁹-His¹⁰-
Asp¹¹-Gly¹²-Tyr¹³-Cys¹⁴-Leu¹⁵-His¹⁶-Asp¹⁷-Gly¹⁸-Val¹⁹-
Cys²⁰-Met²¹-Tyr²²-Ile²³-Glu²⁴-Ala²⁵-Leu²⁶-Asp²⁷-Lys²⁸-
Tyr²⁹-Ala³⁰-Cys³¹-Asn³²-Cys³³-Val³⁴-Val³⁵-Gly³⁶-Tyr³⁷-
Ile³⁸-Gly³⁹-Glu⁴⁰-Arg⁴¹-Cys⁴²-Gln⁴³-Tyr⁴⁴-Arg⁴⁵-Asp⁴⁶-
Leu⁴⁷-Lys⁴⁸-Trp⁴⁹-Trp⁵⁰-Glu⁵¹-Leu⁵²-Arg⁵³-OH

or a variant thereof,

and said process comprises:
coupling a first peptide fragment comprising (or more preferably consisting of) the sequence:
PG₁-Asn¹-Ser²-Asp³-Ser⁴-Glu⁵-Cys⁶-Pro⁷-Leu⁸-ψSer⁹-His¹⁰-Asp¹¹-Gly¹²-OH [SEQ ID NO: 4] or a variant thereof;
   wherein:
      PG₁ is an N-terminal protecting group selected from Boc and Fmoc; and
      the C-terminal amino acid is optionally in the form of an activated carboxylic acid derivative;
in solution with a second peptide fragment (or more preferably consisting of) the sequence:
H-Tyr¹³-Cys¹⁴-Leu¹⁵-His¹⁶-Asp¹⁷-Gly¹⁸-Val¹⁹-Cys²⁰-Met²¹-Tyr²²-Ile²³-Glu²⁴-Ala²⁵-Leu²⁶-Asp²⁷-Lys²⁸-Tyr²⁹-Ala³⁰-Cys³¹-Asn³²-Cys³³-Val³⁴-Val³⁵-Gly³⁶-Tyr³⁷-Ile³⁸-Gly³⁹-Glu⁴⁰-Arg⁴¹-Cys⁴²-Gln⁴³-Tyr⁴⁴-Arg⁴⁵-Asp⁴⁶-Leu⁴⁷-Lys⁴⁸-Trp⁴⁹-Trp⁵⁰-Glu⁵¹-Leu⁵²-Arg⁵³-O-PG₂ [SEQ ID NO: 5] or a variant thereof;
wherein PG₂ is a protecting group selected from chlorotrityl and t-butyl;
and wherein one or more of the amino acid residues in said first and second peptide fragments is optionally protected, preferably with an acid-cleavable protecting group.

In one preferred embodiment, the EGF-like peptide comprises (or more preferably consists of) the following sequence:

H-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-Tyr¹³(P)-Cys¹⁴(P)-Leu¹⁵-His¹⁶(P)-Asp¹⁷(P)-Gly¹⁸-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-OH [SEQ ID NO: 9] or a variant thereof,

wherein the first peptide fragment comprises (or more preferably consists of) the following sequence:

PG₁-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-OH [SEQ ID NO: 20] or a variant thereof;

and the second peptide fragment comprises (or more preferably consists of) the following sequence:

H-Tyr¹³(P)-Cys¹⁴(P)-Leu¹⁵-His¹⁶(P)-Asp¹⁷(P)-Gly¹⁸-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 32] or a variant thereof;

wherein each P represents a side chain protecting group which may be the same or different.

In one preferred embodiment,the EGF-like peptide comprises (or more preferably consists of) the following sequence:

H-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-Tyr¹³(tBu)-Cys¹⁴(Trt)-Leu¹⁵-His¹⁶(Trt)-Asp¹⁷(tBu)-Gly¹⁸-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-OH [SEQ ID NO: 12] or a variant thereof;

and wherein the first peptide fragment comprises (or more preferably consists of) the following sequence:

PG₁-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-OH [SEQ ID NO: 18] or a variant thereof, and the second peptide fragment comprises (or more preferably consists of) the following sequence:

H-Tyr¹³(tBu)-Cys¹⁴(Trt)-Leu¹⁵-His¹⁶(Trt)-Asp¹⁷(tBu)-Gly¹⁸-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 33] or a variant thereof.

In one preferred embodiment, the first peptide fragment PG₁-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-OH [SEQ ID NO: 34] or PG₁-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-OH [SEQ ID NO: 18] is prepared by solid phase peptide synthesis starting with Fmoc-Gly-OH.

In one preferred embodiment, the second peptide fragment H-Tyr¹³(P)-Cys¹⁴(P)-Leu¹⁵-His¹⁶(P)-Asp¹⁷(P)-Gly¹⁸-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(D)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 32] or H-Tyr¹³(tBu)-Cys¹⁴(Trt)-Leu¹⁵-His¹⁶(Trt)-Asp¹⁷(tBu)-Gly¹⁸-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 33] is prepared by solid phase peptide synthesis starting with Fmoc-Arg(P) or Fmoc-Arg(Pbf).

In one preferred embodiment, the second peptide fragment H-Tyr¹³(P)-Cys¹⁴(P)-Leu¹⁵-His¹⁶(P)-Asp¹⁷(P)-Gly¹⁸-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 32] is prepared by fragment condensation of PG₁-Tyr¹³(P)-Cys¹⁴(P)-Leu¹⁵-His¹⁶(P)-Asp¹⁷(P)-Gly¹⁸-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-OH [SEQ ID NO: 35] and H-Tyr37(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 23]. Preferably, the protecting group PG₁ is then removed.

In one preferred embodiment, the second peptide fragment H-Tyr¹³(tBu)-Cys¹⁴(Trt)-Leu¹⁵-His¹⁶(Trt)-Asp¹⁷(tBu)-Gly¹⁸-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 33] is prepared by fragment condensation of PG₁-Tyr¹³(tBu)-Cys¹⁴(Trt)-Leu¹⁵-His¹⁶(Trt)-Asp¹⁷(tBu)-Gly¹⁸-Val¹⁹-Cys²⁰(Trt)-Met²¹-Tyr²²(tBu)-Ile²³-Glu24(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-OH [SEQ ID NO: 36] and H-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 25]. Preferably, the protecting group PG₁ is then removed.

In one preferred embodiment, the peptide fragment PG₁-Tyr¹³(P)-Cys¹⁴(P)-Leu¹⁵-His¹⁶(P)-Asp¹⁷(P)-Gly¹⁸-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-OH [SEQ ID NO: 35] or PG₁-Tyr¹³(tBu)-Cys¹⁴(Trt)-Leu¹⁵-His¹⁶(Trt)-Asp¹⁷(tBu)-Gly¹⁸-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-OH [SEQ ID NO: 36] is prepared by solid phase peptide synthesis starting with Fmoc-Gly-OH.

In one preferred embodiment, the peptide fragment H-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 23] or H-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 25] is prepared by solid phase peptide synthesis starting with Fmoc-Arg(P) or Fmoc-Arg(Pbf).

In one preferred embodiment the invention relates to a process for preparing EGF(1-53), said process comprising the steps of:
(a) coupling a fragment Fmoc-EGF(19-36)-OH with a fragment H-EGF(37-53)-OClt to form Fmoc-EGF(19-53)-OClt;
(b) removing the FMoc group from the fragment Fmoc-EGF(19-53)-OClt to form H-EGF(19-53)-OClt;
(c) coupling said fragment H-EGF(19-53)-OClt with a fragment Boc-EGF(1-18)-OH to form the protected linear peptide Boc-EGF(1-53)-OClt;
(d) treating the protected linear peptide Boc-EGF(1-53)-OClt with a solution of iodine in TFA/dichloromethane;
(e) globally deprotecting the product of step (d) by treating with TFA/H₂O/DTT to form crude linear peptide EGF(1-53);
(f) treating the product of step (e) with DMSO and a water solution containing Tris and guanidine hydrochloride;
(g) optionally purifying the product of step (f) by HPLC.

Preferably, the fragment Fmoc-EGF(19-36)-OH in step (a) is activated, for example, by treating with HOBt.H₂O.

### Process for Preparing Transforming Growth Factor-α (TGF-α)

In one preferred embodiment, the EGF-like peptide is transforming growth factor-α (TGF-α), or an analogue or variant thereof.

In one preferred embodiment, the EGF-like peptide is human transforming growth factor-α (hTGF-α), or an analogue or variant thereof.

In one preferred embodiment, the EGF-like peptide comprises (or more preferably consists of) the following sequence:

H-Val¹-Val²-Ser³-His⁴-Phe⁵-Asn⁶-Asp⁷-Cys⁸-Pro⁹-Asp¹⁰-Ser¹¹-His¹²-Thr¹³-Gln¹⁴-Phe¹⁵-Cys¹⁶-Phe¹⁷-His¹⁸-Gly¹⁹-Thr²⁰-Cys²¹-Arg²²-Phe²³-Leu²⁴-Val²⁵-Gln²⁶-Glu²⁷-Asp²⁸-Lys²⁹-Pro³⁰-Ala³¹-Cys³²-Val³³-Cys³⁴-His³⁵-Ser³⁶-Gly³⁷-Tyr³⁸-Val³⁹-Gly⁴⁰-Ala⁴¹-Arg⁴²-Cys⁴³-Glu⁴⁴-His⁴⁵-Ala⁴⁶-Asp⁴⁷-Leu⁴⁸-Leu⁴⁹-Ala⁵⁰-OH [SEQ ID NO: 6] or a variant thereof;

and said process comprises:
coupling a first peptide fragment comprising (or more preferably consisting of) the sequence:
PG₁-Val¹-Val²-Ser³-His⁴-Phe⁵-Asn⁶-Asp⁷-Cys⁸-Pro⁹-Asp¹⁰-Ser¹¹-His¹²-Thr¹³-Gln¹⁴-Phe¹⁵-Cys¹⁶-Phe¹⁷-His¹⁸-Gly¹⁹-OH [SEQ ID NO: 7] or a variant thereof;
   wherein:
      PG₁ is an N-terminal protecting group selected from Boc and Fmoc; and
      the C-terminal amino acid is optionally in the form of an activated carboxylic acid derivative;
in solution with a second peptide fragment comprising (or more preferably consisting of) the sequence:
H-Thr²⁰-Cys²¹-Arg²²-Phe²³-Leu²⁴-Val²⁵-Gln²⁶-Glu²⁷-Asp²⁸-Lys²⁹-Pro³⁰-Ala³¹-Cys³²-Val³³-Cys³⁴-His³⁵-Ser³⁶-Gly³⁷-Tyr³⁸-Val³⁹-Gly⁴⁰-Ala⁴¹-Arg⁴²-Cys⁴³-Glu⁴⁴-His⁴⁵-Ala⁴⁶-Asp⁴⁷-Leu⁴⁸-Leu⁴⁹-Ala⁵⁰-O-PG₂ [SEQ ID NO: 8] or a variant thereof,
wherein PG₂ is a protecting group selected from chlorotrityl and t-butyl;
and wherein one or more of the amino acid residues in said first and second peptide fragments is optionally protected, preferably with an acid-cleavable protecting group.

In one preferred embodiment, the EGF-like peptide comprises (or more preferably consists of) the following sequence:

PG₁-Val¹-Val²-Ser³(P)-His⁴(P)-Phe⁵-Asn⁶(P)-Asp⁷(P)-Cys⁸(P)-Pro⁹-Asp¹⁰(P)-ψSer¹¹-His¹²(P)-Thr¹³(P)-Gln¹⁴(P)-Phe¹⁵-Cys¹⁶(P)-Phe¹⁷-His¹⁸(P)-Gly¹⁹-Thr²⁰(P)-Cys²¹(P)-Arg²²(P)-Phe²³-Leu²⁴-Val²⁵-Gln²⁶-Glu²⁷(P)-Asp²⁸(P)-Lys²⁹(P)-Pro³⁰-Ala³¹-Cys³²(P)-Val³³-Cys³⁴(P)-His³⁵-Ser³⁶(P)-Gly³⁷-Tyr³⁸(P)-Val³⁹-Gly⁴⁰-Ala⁴¹-Arg⁴²(P)-Cys⁴³(P)-Glu⁴⁴(P)-His⁴⁵(P)-Ala⁴⁶-Asp⁴⁷(P)-Leu⁴⁸-Leu⁴⁹-Ala⁵⁰-O-PG₂ [SEQ ID NO: 37] or a variant thereof, wherein the first peptide fragment comprises (or more preferably consists of) the following sequence:

PG₁-Val¹-Val²-Ser³(P)-His⁴(P)-Phe⁵-Asn⁶(P)-Asp⁷(P)-Cys⁸(P)-Pro⁹-Asp¹⁰(P)-ψSer¹¹-His¹²(P)-Thr¹³(P)-Gln¹⁴(P)-Phe¹⁵-Cys¹⁶(P)-Phe¹⁷-His¹⁸(P)-Gly¹⁹-OH [SEQ ID NO: 38] or a variant thereof,

and the second peptide fragment comprises (or more preferably consists of) the following sequence:

H-Thr²⁰(P)-Cys²¹(P)-Arg²²(P)-Phe²³-Leu²⁴-Val²⁵-Gln²⁶-Glu²⁷(P)-Asp²⁸(P)-Lys²⁹(P)-Pro³⁰-Ala³¹-Cys³²(P)-Val³³-Cys³⁴(P)-His³⁵-Ser³⁶(P)-Gly³⁷-Tyr³⁸(P)-Val³⁹-Gly⁴⁰-Ala⁴¹-Arg⁴²(P)-Cys⁴³(P)-Glu⁴⁴(P)-His⁴⁵(P)-Ala⁴⁶-Asp⁴⁷(P)-Leu⁴⁸-Leu⁴⁹-Ala⁵⁰-O-PG₂ [SEQ ID NO: 39] or a variant thereof,

wherein each P represents a side chain protecting group which may be the same or different.

In one preferred embodiment, the EGF-like peptide comprises (or more preferably consists of) the following sequence:

PG₁-Val¹-Val²-Ser³(tBu)-His⁴(Trt)-Phe⁵-Asn⁶(Trt)-Asp⁷(tBu)-Cys⁸(Trt)-Pro⁹-Asp¹⁰(tBu)-ψSer¹¹-His¹²(Trt)-Thr¹³(tBu)-Gln¹⁴(Trt)-Phe¹⁵-Cys¹⁶(Trt)-Phe¹⁷-His¹⁸(Trt)-Gly¹⁹-Thr²⁰(tBu)-Cys²¹(Trt)-Arg²²(Pbf)-Phe²³-Leu²⁴-Val²⁵-Gln²⁶-Glu²⁷(tBu)-Asp²⁸(tBu)-Lys²⁹(Boc)-Pro³⁰-Ala³¹-Cys³²(Trt)-Val³³-Cys³⁴(Trt)-His³⁵-Ser³⁶(tBu)-Gly³⁷-Tyr³⁸(tBu)-Val³⁹-Gly⁴⁰-Ala⁴¹-Arg⁴²(Pbf)-Cys⁴³(Trt)-Glu⁴⁴(tBu)-His⁴⁵(Trt)-Ala⁴⁶-Asp⁴⁷(tBu)-Leu⁴⁸-Leu⁴⁹-Ala⁵⁰-O-PG₂ [SEQ ID NO: 40] or a variant thereof, wherein the first peptide fragment comprises (or more preferably consists of) the following sequence:

PG₁-Val¹-Val²-Ser³(tBu)-His⁴(Trt)-Phe⁵-Asn⁶(Trt)-Asp⁷(tBu)-Cys⁸(Trt)-Pro⁹-Asp¹⁰(tBu)-ψSer¹¹-His¹²(Trt)-Thr¹³(tBu)-Gln¹⁴(Trt)-Phe¹⁵-Cys¹⁶(Trt)-Phe¹⁷-His¹⁸(Trt)-Gly¹⁹-OH [SEQ ID NO: 41] or a variant thereof,

and the second peptide fragment comprises (or more preferably consists of) the following sequence:

H-Thr²⁰tBu)-Cys²¹(Trt)-Arg²²(Pbf)-Phe²³-Leu²⁴-Val²⁵-Gln²⁶-Glu²⁷(tBu)-Asp²⁸(tBu)-Lys²⁹(Boc)-Pro³⁰-Ala³¹-Cys³²(Trt)-Val³³-Cys³⁴(Trt)-His³⁵-Ser³⁶(tBu)-Gly³⁷-Tyr³⁸(tBu)-Val³⁹-Gly⁴⁰-Ala⁴¹-Arg⁴²(Pbf)-Cys⁴³(Trt)-Glu⁴⁴(tBu)-His⁴⁵(Trt)-Ala⁴⁶-Asp⁴⁷(tBu)-Leu⁴⁸-Leu⁴⁹-Ala⁵⁰-O-PG₂ [SEQ ID NO: 42] or a variant thereof.

In one preferred embodiment, the first peptide fragment PG₁-Val¹-Val²-Ser³(P)-His⁴(P)-Phe⁵-Asn⁶(P)-Asp⁷(P)-Cys⁸(P)-Pro⁹-Asp¹⁰(P)-ψSer¹¹-His¹²(P)-Thr¹³(P)-Gln¹⁴(P)-Phe¹⁵-Cys¹⁶(P)-Phe¹⁷-His¹⁸(P)-Gly¹⁹-OH [SEQ ID NO: 38] or PG₁-Val¹-Val²-Ser³(tBu)-His⁴(Trt)-Phe⁵-Asn⁶(Trt)-Asp⁷(tBu)-Cys⁸(Trt)-Pro⁹-Asp¹⁰(tBu)-ψSer¹¹-His¹²(Trt)-Thr¹³(tBu)-Gln¹⁴(Trt)-Phe¹⁵-Cys¹⁶(Trt)-Phe¹⁷-His¹⁸(Trt)-Gly¹⁹-OH [SEQ ID NO: 41] is prepared by solid phase peptide synthesis starting with Fmoc-Gly-OH.

In one preferred embodiment, the second peptide fragment comprising (or more preferably consisting of) the sequence H-Thr²⁰(P)-Cys²¹(P)-Arg²²(P)-Phe²³-Leu²⁴-Val²⁵-Gln²⁶-Glu²⁷(P)-Asp²⁸(P)-Lys²⁹(P)-Pro³⁰-Ala³¹-Cys³²(P)-Val³³-Cys³⁴(P)-His³⁵-Ser³⁶(P)-Gly³⁷-Tyr³⁸(P)-Val³⁹-Gly⁴⁰-Ala⁴¹-Arg⁴²(P)-Cys⁴³(P)-Glu⁴⁴(P)-His⁴⁵(P)-Ala⁴⁶-Asp⁴⁷(P)-Leu⁴⁸-Leu⁴⁹-Ala⁵⁰-O-PG₂ [SEQ ID NO: 39] is prepared by fragment condensation of PG₁-Thr²⁰P)-Cys²¹(P)-Arg²²(P)-Phe²³-Leu²⁴-Val²⁵-Gln²⁶-Glu²⁷(P)-Asp²⁸(P)-Lys²⁹(P)-Pro³⁰-Ala³¹-Cys³²(P)-Val³³-Cys³⁴(P)-His³⁵-Ser³⁶(P)-Gly³⁷-OH [SEQ ID NO: 43] and H-Tyr³⁸(P)-Val³⁹-Gly⁴⁰-Ala⁴¹-Arg⁴²(P)-Cys⁴³(P)-Glu⁴⁴(P)-His⁴⁵(P)-Ala⁴⁶-Asp⁴⁷(P)-Leu⁴⁸-Leu⁴⁹-Ala⁵⁰-O-PG₂ [SEQ ID NO: 44]. Preferably, the protecting group PG₁ is then removed.

In one preferred embodiment, the second peptide fragment comprising (or more preferably consisting of) the sequence H-Thr²⁰(tBu)-Cys²¹(Trt)-Arg²²(Pbf)-Phe²³-Leu²⁴-Val²⁵-Gln²⁶-Glu²⁷(tBu)-Asp²⁸(tBu)-Lys²⁹(Boc)-Pro³⁰-Ala³¹-Cys³²(Trt)-Val³³-Cys³⁴(Trt)-His³⁵-Ser³⁶(tBu)-Gly³⁷-Tyr³⁸(tBu)-Val³⁹-Gly⁴⁰-Ala⁴¹-Arg⁴²(Pbf)-Cys⁴³(Trt)-Glu⁴⁴(tBu)-His⁴⁵(Trt)-Ala⁴⁶-Asp⁴⁷(tBu)-Leu⁴⁸-Leu⁴⁹-Ala⁵⁰-O-PG₂ [SEQ ID NO: 45] is prepared by fragment condensation of PG₁-Thr²⁰(tBu)-Cys²¹(Trt)-Arg²²(Pbf)-Phe²³-Leu²⁴-Val²⁵-Gln²⁶-Glu²⁷(tBu)-Asp²⁸(tBu)-Lys²⁹(Boc)-Pro³⁰-Ala³¹-Cys³²(Trt)-Val³³-Cys³⁴(Trt)-His³⁵-Ser³⁶(tBu)-Gly³⁷-OH [SEQ ID NO: 46] and H-Tyr³⁸(tBu)-Val³⁹-Gly⁴⁰-Ala⁴¹-Arg⁴²(Pbf)-Cys⁴³-(Trt)-Glu⁴⁴(tBu)-His⁴⁵(Trt)-Ala⁴⁶-Asp⁴⁷(tBu)-Leu⁴⁸-Leu⁴⁹-Ala⁵⁰-O-PG₂ [SEQ ID NO: 47]. Preferably, the protecting group PG₁ is then removed.

In one preferred embodiment, the peptide fragment H-Tyr³⁸(P)-Val³⁹-Gly⁴⁰-Ala⁴¹-Arg⁴²(P)-Cys⁴³(P)-Glu⁴⁴(P)-His⁴⁵(P)-Ala⁴⁶-Asp⁴⁷(P)-Leu⁴⁸-Leu⁴⁹-Ala⁵⁰-O-PG₂ [SEQ ID NO: 48] or H-Tyr³⁸(tBu)-Val³⁹-Gly⁴⁰-Ala⁴¹-Arg⁴²(Pbf)-Cys⁴³(Trt)-Glu⁴⁴(tBu)-His⁴⁵(Trt)-Ala⁴⁶-Asp⁴⁷(tBu)-Leu⁴⁸-Leu⁴⁹-Ala⁵⁰-O-PG₂ [SEQ ID NO: 47] is prepared by solid phase peptide synthesis starting with Fmoc-Ala-OH.

In one preferred embodiment, the second peptide fragment PG₁-Thr²⁰(P)-Cys²¹(P)-Arg²²(P)-Phe²³-Leu²⁴-Val²⁵-Gln²⁶-Glu²⁷(P)-Asp²⁸(P)-Lys²⁹(P)-Pro³⁰-Ala³¹-Cys³²(P)-Val³³-Cys³⁴(P)-His³⁵-Ser³⁶(P)-Gly³⁷-OH [SEQ ID NO: 43] or PG₁-Thr²⁰(tBu)-Cys²¹(Trt)-Arg²²(Pbf)-Phe²³-Leu²⁴-Val²⁵-Gln²⁶-Glu²⁷(tBu)-Asp²⁸(tBu)-Lys²⁹(Boc)-Pro³⁰-Ala³¹-Cys³²(Trt)-Val³³-Cys³⁴(Trt)-His³⁵-Ser³⁶(tBu)-Gly³⁷-OH [SEQ ID NO: 46] is prepared by solid phase peptide synthesis starting with Fmoc-Gly-OH.

In one preferred embodiment the invention relates to a process for preparing TGF(1-50), said process comprising the steps of:
(a) coupling a fragment Fmoc-TGF(20-37)-OH with a fragment H-TGF(38-50)-OClt to form Fmoc-EGF(20-50)-OClt;
(b) removing the FMoc group from the fragment Fmoc-TGF(20-50)-OClt to form H-TGF(20-50)-OClt;
(c) coupling said fragment H-TGF(20-50)-OClt with a fragment Boc-TGF(1-19)-OH to form the protected linear peptide Boc-TGF(1-50)-OClt;
(d) treating the protected linear peptide Boc-TGF(1-50)-OClt with a solution of iodine in TFA/dichloromethane;
(e) globally deprotecting the product of step (d) by treating with TFA/H₂O/DTT to form crude linear peptide TGF(1-50);
(f) treating the product of step (e) with DMSO and a water solution containing Tris and guanidine hydrochloride;
(g) optionally purifying the product of step (f) by HPLC.

Preferably, the fragment Fmoc-TGF(20-37)-OH in step (a) is activated, for example, by treating with HOBt.H₂O.

For all of the embodiments described herein, preferably PG₂ is chlorotrityl or trityl, more preferably, chlorotrityl. Advantageously, the use of a chlorotrityl protecting group in the synthesis leads to a significant increase in the overall yield. For example, in some instances, using a chlorotrityl protecting group can lead to an overall increase in yield of the desired peptide of as much as 25%.

In one preferred embodiment, PG₁ is Boc (butyloxycarbonyl).

For all the embodiments described herein, preferably, the first fragment is prepared on solid phase or in solution. Where the first fragment is prepared on solid phase, it is cleaved from the resin before coupling with the second fragment in solution.

For all the embodiments described herein, preferably the second fragment is prepared on solid phase or in solution. Where the second fragment is prepared on solid phase, it is cleaved from the resin before coupling with the first fragment in solution.

For all the embodiments described herein, preferably the second fragment is prepared by coupling two or more sub-fragments.

In one preferred embodiment, the crude EGF-like peptide is purified by preparative HPLC using various buffers in water/acetonitrile or water/methanol.

Another aspect of the invention relates to the use of one or more peptide fragments as described herein in the synthesis of an EGF-like peptide or analogue or variant thereof, more preferably, EGF or TGF-α.

### Synthesis of EGF by Specific Fragment Combination

A second aspect of the invention relates to a process for preparing an EGF-like peptide having the following sequence:

[SEQ ID NO: 1]
H-Asn¹-Ser²-Asp³-Ser⁴-Glu⁵-Cys⁶-Pro⁷-Leu⁸-Ser⁹-His¹⁰-
Asp¹¹-Gly¹²-Tyr¹³-Cys¹⁴-Leu¹⁵-His¹⁶-Asp¹⁷-Gly¹⁸-Val¹⁹-
Cys²⁰-Met²¹-Tyr²²-Ile²³-Glu²⁴-Ala²⁵-Leu²⁶-Asp²⁷-Lys²⁸-
Tyr²⁹-Ala³⁰-Cys³¹-Asn³²-Cys³³-Val³⁴-Val³⁵-Gly³⁶-Tyr³⁷-
Ile³⁸-Gly³⁹-Glu⁴⁰-Arg⁴¹-Cys⁴²-Gln⁴³-Tyr⁴⁴-Arg⁴⁵-Asp⁴⁶-
Leu⁴⁷-Lys⁴⁸-Trp⁴⁹-Trp⁵⁰-Glu⁵¹-Leu⁵²-Arg⁵³-OH

or a variant thereof,

wherein said process comprises:
coupling a first peptide fragment comprising (or preferably consisting of) the sequence:
PG₁-Asn¹-Ser²-Asp³-Ser⁴-Glu⁵-Cys⁶-Pro⁷-Leu⁸-Ser⁹-His¹⁰-Asp¹¹-Gly¹²-Tyr¹³-Cys¹⁴-Leu¹⁵-His¹⁶-Asp¹⁷-Gly¹⁸-OH [SEQ ID NO: 2] or a variant thereof,
   wherein:
      PG₁ is an N-terminal protecting group, preferably selected from Boc and Fmoc; and
      the C-terminal amino acid is optionally in the form of an activated carboxylic acid derivative;
in solution with a second peptide fragment (or preferably consisting of) the sequence:
H-Val¹⁹-Cys²⁰-Met²¹-Tyr²²-Ile²³-Glu²⁴-Ala²⁵-Leu²⁶-Asp²⁷-Lys²⁸-Tyr²⁹-Ala³⁰-Cys³¹-Asn³²-Cys³³-Val³⁴-Val³⁵-Gly³⁶-Tyr³⁷-Ile³⁸-Gly³⁹-Glu⁴⁰-Arg⁴¹-Cys⁴²-Gln⁴³-Tyr⁴⁴-Arg⁴⁶-Asp⁴⁶-Leu⁴⁷-Lys⁴⁸-Trp⁴⁹-Trp⁵⁰-Glu⁵¹-Leu⁵²-Arg⁵³-O-PG₂ [SEQ ID NO: 3] or a variant thereof,
wherein PG₂ is a C-terminal protecting group, preferably selected from chlorotrityl and t-butyl;
and wherein one or more of the amino acid residues in said first and second peptide fragments is optionally protected, preferably with an acid-cleavable protecting group; and
optionally removing protecting groups PG₁ and PG₂.

A third aspect of the invention relates to a process for preparing an EGF-like peptide comprising (or preferably consisting of) the following sequence:

[SEQ ID NO: 1]
H-Asn¹-Ser²-Asp³-Ser⁴-Glu⁵-Cys⁶-Pro⁷-Leu⁸-Ser⁹-His¹⁰-
Asp¹¹-Gly¹²-Tyr¹³-Cys¹⁴-Leu¹⁵-His¹⁶-Asp¹⁷-Gly¹⁸-Val¹⁹-
Cys²⁰-Met²¹-Tyr²²-Ile²³-Glu²⁴-Ala²⁵-Leu²⁶-Asp²⁷-Lys²⁸-
Tyr²⁹-Ala³⁰-Cys³¹-Asn³²-Cys³³-Val³⁴-Val³⁵-Gly³⁶-Tyr³⁷-
Ile³⁸-Gly³⁹-Glu⁴⁰-Arg⁴¹-Cys⁴²-Gln⁴³-Tyr⁴⁴-Arg⁴⁵-Asp⁴⁶-
Leu⁴⁷-Lys⁴⁸-Trp⁴⁹-Trp⁵⁰-Glu⁵¹-Leu⁵²-Arg⁵³-OH

or a variant thereof,

wherein said process comprises:
coupling a first peptide fragment comprising (or preferably consisting of) the sequence:
PG₁-Asn¹-Ser²-Asp³-Ser⁴-Glu⁵-Cys⁶-Pro⁷-Leu⁸-ψSer⁹-His¹⁰-Asp¹¹-Gly¹²-OH [SEQ ID NO: 4] or a variant thereof;
   wherein:
      PG₁ is an N-terminal protecting group, preferably selected from Boc and Fmoc; and
      the C-terminal amino acid is optionally in the form of an activated carboxylic acid derivative;
in solution with a second peptide fragment comprising (or preferably consisting of) the sequence:
H-Tyr¹³-Cys¹⁴-Leu¹⁵-His¹⁶-Asp¹⁷-Gly¹⁸-Val¹⁹-Cys²⁰-Met²¹-Tyr²²-Ile²³-Glu²⁴-Ala²⁵-Leu²⁶-Asp²⁷-Lys²⁸-Tyr²⁹-Ala³⁰-Cys³¹-Asn³²-Cys³³-Val³⁴-Val³⁵-Gly³⁶-Tyr³⁷-Ile³⁸-Gly³⁹-Glu⁴⁰-Arg⁴¹-Cys⁴²-Gln⁴³-Tyr⁴⁴-Arg⁴⁵-Asp⁴⁶-Leu⁴⁷-Lys⁴⁸-Trp⁴⁹-Trp⁵⁰-Glu⁵¹-Leu⁵²-Arg⁵³-O-PG₂ [SEQ ID NO: 5] or a variant thereof;
wherein PG₂ is a C-terminal protecting group, preferably selected from chlorotrityl and t-butyl;
and wherein one or more of the amino acid residues in said first and second peptide fragments is optionally protected, preferably with an acid-cleavable protecting group; and
optionally removing protecting groups PG₁ and PG₂.

### Synthesis of TGF-α by Specific Fragment Combination

A fourth aspect of the invention relates to a process for preparing an EGF-like peptide comprising (or preferably consisting of) the following sequence:

H-Val¹-Val²-Ser³-His⁴-Phe⁵-Asn⁶-Asp⁷-Cys⁸-Pro⁹-Asp¹⁰-Ser¹¹-His¹²-Thr¹³-Gln¹⁴-Phe¹⁵-Cys¹⁶-Phe¹⁷-His¹⁸-Gly¹⁹-Thr²⁰-Cys²¹-Arg²²-Phe²³-Leu²⁴-Val²⁵-Gln²⁶-Glu²⁷-Asp²⁸-Lys²⁹-Pro³⁰-Ala³¹-Cys³²-Val³³-Cys³⁴-His³⁵-Ser³⁶-Gly³⁷-Tyr³⁸-Val³⁹-Gly⁴⁰-Ala⁴¹-Arg⁴²-Cys⁴³-Glu⁴⁴-His⁴⁵-Ala⁴⁶-Asp⁴⁷-Leu⁴⁸-Leu⁴⁹-Ala⁵⁰-OH [SEQ ID NO: 6] or a variant thereof;

wherein said process comprises:
coupling a first peptide fragment having the sequence:
PG₁-Val¹-Val²-Ser³-His⁴-Phe⁵-Asn⁶-Asp⁷-Cys⁸-Pro⁹-Asp¹⁰-Ser¹¹-His¹²-Thr¹³-Gln¹⁴-Phe¹⁵-Cys¹⁶-Phe¹⁷-His¹⁸-Gly¹⁹-OH [SEQ ID NO: 7] or a variant thereof;
   wherein:
      PG₁ is an N-terminal protecting group, preferably selected from Boc and Fmoc; and
      the C-terminal amino acid is optionally in the form of an activated carboxylic acid derivative;
in solution with a second peptide fragment having the sequence:
   H-Thr²⁰-Cys²¹-Arg²²-Phe²³-Leu²⁴-Val²⁵-Gln²⁶-Glu²⁷-Asp²⁸-Lys²⁹-Pro³⁰-Ala³¹-Cys³²-Val³³-Cys³⁴-His³⁵-Ser³⁶-Gly³⁷-Tyr³⁸-Val³⁹-Gly⁴⁰-Ala⁴¹-Arg⁴²-Cys⁴³-Glu⁴⁴-His⁴⁵-Ala⁴⁶-Asp⁴⁷-Leu⁴⁸-Leu⁴⁹-Ala⁵⁰-O-PG₂ [SEQ ID NO: 8] or a variant thereof,
   wherein PG₂ is a C-terminal protecting group, preferably selected from chlorotrityl and t-butyl;
   and wherein one or more of the amino acid residues in said first and second peptide fragments is optionally protected, preferably with an acid-cleavable protecting group; and
   optionally removing protecting groups PG₁ and PG₂.

For the above-mentioned second, third and fourth aspects of the invention, preferably, the process in each case further comprises subjecting the protected or unprotected linear EGF-like peptide formed in the fragment condensation step to certain conditions to form the tertiary structure of the EGF-like peptide. Preferred conditions include steps (IV)(a), (IV)(b) and (V) as set out above for the first aspect.

Thus, in one embodiment, for the above-mentioned second, third and fourth aspects of the invention, preferably the process comprises the steps of: (III) coupling the C-terminal amino acid of said first peptide fragment with the N-terminal amino acid of said second peptide fragment in solution to form a linear protected EGF-like peptide;

(IV)(a)
(i) treating the linear protected EGF-like peptide formed in step (III) with iodine to form an oxidized mixture;
(ii) globally deprotecting the oxidized mixture obtained in step (IV)(a)(i) by treating with trifluoroacetic acid (TFA);
(iii) treating the deprotected oxidized mixture obtained in step (IV)(a)(ii) with DMSO/DTT to form a crude EGF-like peptide; or

(IV)(b)
(i) globally deprotecting the linear protected EGF-like peptide obtained in step (III) by treating with trifluoroacetic acid (TFA);
(ii) treating the deprotected mixture obtained in step (IV)(b)(i) with DMSO to form a crude EGF-like peptide; and

(V) optionally purifying the crude EGF-like peptide.

In one particularly preferred embodiment, the process comprises the steps of:
coupling the C-terminal amino acid of said first peptide fragment with the N-terminal amino acid of said second peptide fragment in solution to form a protected EGF-like peptide;
treating the protected EGF-like peptide formed in step (a) with iodine to form an oxidized mixture;
globally deprotecting the oxidized mixture obtained in step (iv) by treating with trifluoroacetic acid (TFA);
treating the deprotected oxidized mixture obtained in step (v) with dithiothreitol (DTT) and DMSO to form a crude EGF-like peptide; and
optionally purifying the crude EGF-like peptide.

For the above-mentioned second, third and fourth aspects of the invention, preferred embodiments for each of the process steps are as set out above for the first aspect of the invention.

For the above-mentioned second, third and fourth aspects of the invention, suitable N-terminal and C-terminal protecting groups for amino acids will be familiar to the skilled person. Examples may be found in T. W. Greene & P. G. M. Wuts, Protective Groups in Organic Synthesis (2nd edition) J. Wiley & Sons, 1991; and P. J. Kocienski, Protecting Groups, Georg Thieme Verlag, 1994.

Examples of preferred N-terminal protecting groups for amino acids include, but are not limited to, Boc (tert-butyloxycarbonyl) and Fmoc (9-fluorenylmethyloxy-carbonyl). Their lability is caused by the carbamate group which readily releases CO₂ for an irreversible decoupling step. Another suitable carbamate based group is the benzyloxy-carbonyl (Z or Cbz) group; this is removed in harsher conditions. Boc and Fmoc are particularly preferred.

Examples of C-terminal protecting groups for amino acids include chlorotrityl and t-butyl. Chlorotrityl is particularly preferred.

For the above-mentioned second, third and fourth aspects of the invention, preferably in one embodiment the first peptide fragment is prepared by coupling two or more peptide sub-fragments. Preferably, the sub-fragments are as set out above for the first aspect of the invention.

For the above-mentioned second, third and fourth aspects of the invention, preferably in one embodiment the first peptide fragment is prepared by solid phase peptide synthesis.

For the above-mentioned second, third and fourth aspects of the invention, preferably in one embodiment the second peptide fragment is prepared by coupling two or more peptide sub-fragments. Preferably, the sub-fragments are as set out above for the first aspect of the invention.

For the above-mentioned second, third and fourth aspects of the invention, preferably in one embodiment the second peptide fragment is prepared by solid phase peptide synthesis.

For each of the above-described coupling reactions, preferably the COOH group is activated, for example, by treating with HOBt.H₂O.

For the above-mentioned second, third and fourth aspects of the invention, preferred first and second peptide fragments and subfragments thereof, together with methods for the preparation thereof, are as set out above for the first aspect of the invention.

The present invention is further described by way of the following non-limiting examples.

### EXAMPLES

### Abbreviations

Acm acetamidomethyl

AIB (or Aib) 2-aminoisobutyric acid or α-aminoisobutyric acid

Boc or t-Boc t-butyloxycarbonyl

Bt benzotriazole

Bz benzyl

Dde 1-(4,4-Dimethyl-2,6-dioxocyclohex-1-ylidene)ethyl

EDC.HCl 1-ethyl-3-(3′-dimethyl-aminopropyl)carbodiimide hydrochloride#

DMAC dimethyl acetamide

DMF dimethyl fomamide

DPM diphenylmethyl

DCM dichloromethane

DMSO dimethyl sulfoxide

DIC N,N′-Diisopropylcarbodiimide

DIPEA N,N-diisopropylethylamine

HBTU (2-1H-benzotriazol-1yl)-1,1,3,3-tetramethyluronium hexafluoro phosphate, (Hexafluorophosphate Benzotriazole Tetramethyl Uranium)

MeDPM methyl-diphenylmethyl

MeODPM methoxy-diphenylmethyl

MeOH methanol

ivDde 1-(4,4-Dimethyl-2,6-dioxocyclohexylidene)-3-methylbutyl

Fmoc 9-fluorenylmethoxycarbonyl

HPLC High Performance Liquid Chromatography

HOBt Hydroxybenzotriazole

Mmt monomethoxytrityl [(4-methoxyphenyl)diphenylmethyl]

Mtt 4-methyltrityl

NMP N-methylpyrrolidone

Pfp pentafluorophenyl

Su succinimide

tBu tert-butyl

Pal palmitoyl

Pbf 2,2,4,6,7-pentamethyl-dihydrobenzofuran-5-sulfonyl

TBTU N,N,N′,N′-Tetramethyl-O-(benzotriazol-1-Auronium tetrafluoroborate

TFA trifluoroacetic acid

Tris tris(hydroxymethyl)aminomethane

Trt trityl

Clt chlorotrityl

### Experimental Section

Synthesis of Fmoc-EGF(37-53)-OH: 96 g of 2-Chlorotrityl chloride resin are swelled with 700 ml DCM. 56 ml DIPEA and 25.2 g Fmoc-Arg(Pbf)-OH are added. The reaction is left to stand for 3 hours and 28 ml of methanol is added. The resin is filtered and neutralized with 288 ml of DCM/MeOH/DIPEA. The Fmoc-cleavage is performed with the addition of a mixture of 384 ml piperidine/NMP (15%). All couplings were performed with 2.5 mmol excess of Fmoc-amino acids/HOBt/DIC (1:1.2:1.1) in NMP (0.5M). The protected peptide is cleaved from the resin with 1344 ml of a mixture of TFA/DCM (2%). The TFA is extracted with water (3300 ml) and the peptide is precipitated, after condensation, with addition of Hexane (800 ml). Final Yield (104 g, 72%)

Chlorotrityl protection of Fmoc-EGF(37-53)-OH: 80 g of the protected peptide are dissolved with 296 ml DCM and 32 g of 2-chlorotrityl chloride and 36 ml DIPEA are added. The reaction is left to stand for 2 h and monitored by HPLC. DIPEA is extracted from the dichloromethane mixture with 592 ml of 0.1N hydrochloride acid and Fmoc-EGF(37-53)-OClt is precipitated after condensation with 1600 ml of Hexane and dried under vacuum. Fmoc cleavage is performed with 6 mmol excess of piperidine in NMP (168 ml). 504 ml DCM is added in the reaction mixture and piperidine is extracted from the dichloromethane solution with 504 ml of 0.01N hydrochloride acid. H-EGF(37-53)-OClt is precipitated after condensation with 1056 ml of Hexane, washed 6 times with 392 ml diethylether and dried under vacuum. Final yield 78 g (92%)

Synthesis of Fmoc-EGF(19-36)-OH: 42 g of 2-Chlorotrityl chloride resin are swelled with 232 ml DCM. 26 ml DIPEA and 5 g Fmoc-Gly-OH are added. The reaction is left to stand for 3 hours and 14 ml of methanol is added. The resin is filtered and neutralized with 126 ml of DCM/MeOH/DIPEA. The Fmoc-cleavage is performed with the addition of a mixture of 192 ml piperidine/NMP (15%). All couplings were performed with 2.5 mmol excess of Fmoc-amino acids/HOBt/DIC (1:1.2:1.1) in NMP (0.5M). The protected peptide is cleaved from the resin with 588 ml of a mixture of TFA/DCM (2%). The TFA is extracted with water (1450 ml) and the peptide is precipitated, after condensation, with addition of Hexane (400 ml). Final Yield 42 g (82%)

Synthesis of Boc-EGF(1-18)-OH: 42 g of 2-Chlorotrityl chloride resin are swelled with 232 ml DCM. 26 ml DIPEA and 5 g Fmoc-Gly-OH are added. The reaction is left to stand for 3 hours and 14 ml of methanol is added. The resin is filtered and neutralized with 6.3 ml of DCM/MeOH/DIPEA. The Fmoc-cleavage is performed with the addition of a mixture of 192 ml piperidine/NMP (15%). All couplings were performed with 2.5 mmol excess of Fmoc-amino acids/HOBt/DIC (1:1.2:1.1) in NMP (0.5M). The protected peptide is cleaved from the resin with 588 ml of a mixture of TFA/DCM (2%). The TFA is extracted with water (1450 ml) and the peptide is precipitated, after condensation, with addition of Hexane (400 ml). Final Yield 42 g (80%)

Synthesis of protected EGF (1-53): 40 g Fmoc-EGF(19-36)-OH is activated with HOBt.H₂O (2.1 g) in 140 ml NMP and EDAC.HCl (2.4 g) and H-EGF(37-53)-OClt (52 g) is added in the activated protected peptide. The completion of the reaction is monitored by HPLC. When Fmoc-EGF(19-36)-OH is <0.5% by area comparing to the corresponding Fmoc-EGF(19-53)-OClt, 7 ml of piperidine is added for the Fmoc-cleavage. 432 ml DCM is added in the reaction mixture and piperidine is extracted from the dichloromethane solution with 432 ml of 0.01N hydrochloride acid. H-EGF(19-53)-OClt is precipitated after condensation with 1850 ml of Hexane, washed 6 times with 460 ml diethylether and dried under vacuum. Final yield 84.6 g (94%)

In 84 g of protected H-EGF(19-53)-OClt dissolved in 420 ml NMP, the activated Boc-EGF(1-18)-OH (40 g), HOBt.H₂O (1.9 g), EDAC.HCl (2.3 g) dissolved in 310 ml NMP is added. The reaction is monitored by HPLC. When H-EGF(19-53)-OClt is <1% by area comparing to crude EGF the reaction is stopped by the addition of 7.3 Lt of water and filtered. The final protected peptide is washed 3 times with water (400 ml) and dried under vacuum until the water content is <3%. Final Yield 115.2 g (96%)

A similar synthetic strategy can be used by preparing the fragment Fmoc-EGF(13-36)-OH and coupling with the fragment H-EGF(37-53)-OClt to form Fmoc-EGF(13-53)-OClt, followed by removal of the Fmoc group to form H-EGF(13-53)-OClt. H-EGF(13-53)-OClt can then be coupled with Boc-EGF(1-12)-OH to form the protected linear EGF peptide.

Iodine oxidation of protected EGF (1-53): The protected peptide (110 g) is dissolved in 800 ml DCM and an iodine solution (3.7 g) in 800 ml 1% TFA/DCM is added. The reaction is left to stand for 1 h and an aqueous solution (1.6 Lt) of sodium sulfate pentahydrate (7.3 g) is added. The DCM/peptide solution is extracted two more times with 1.6 Lt of water, concentrated under vacuum, precipitated with Hexane (1.2 Lt) and washed 3 times with diethylether (500 ml). Final Yield 95.7 g (99%)

Global Deprotection: 95 g of Boc-EGF(1-53)-OClt is deprotected by its protecting groups by the addition of 5.6 Lt of a mixture consisting of TFA/H₂O/DTT (94:3:3) in RT for 2.5 h. The DTT functions as a scavenger and to avoid possible premature oxidation. The reaction is concentrated under vacuum and crude linear EGF is precipitated by the addition of 940 ml diethylether and washed three times with diethylether (240 ml). Final yield 47.2 g (100%).

DMSO/DTT oxidation: Crude linear EGF (40 g) is dissolved in 5.28 Lt DMSO and a water solution (21.4 Lt) containing Tris (200 g) and guanidine hydrochloride (260 g) is added at RT. The guanidine hydrochloride functions as a chaotrope. The reaction is left to stand for 24 h and its completion is monitored by HPLC. Final Yield 15%.

HPLC Purification: After acidification with 0.2% TFA the crude linear EGF solution is directly loaded on a preparative HPLC column packed with Kromasil C-18, 100A, 13 μm. Crude native h-EGF is purified with a two-step purification process, the first with TFA and the second with ammonium bicarbonate (pH 7.8). Acetonitrile is used as the organic modifier. Fractions containing native EGF with a purity >98% are further lyophilized. Final Yield 1.04 g (70%)

Synthesis of Fmoc-TGF(38-50)-OH: 70 g of 2-Chlorotrityl chloride resin are swelled with 350 ml DCM. 48 ml DIPEA and 10 g Fmoc-Ala-OH are added. The reaction is left to stand for 3 hours and 21 ml of methanol is added. The resin is filtered and neutralized with 250 ml of DCM/MeOH/DIPEA. The Fmoc-cleavage is performed with the addition of a mixture of 230 ml piperidine/NMP (15%). All couplings were performed with 2.5 mmol excess of Fmoc-amino acids/HOBt/DIC (1:1.2:1.1) in NMP (0.5M). The protected peptide is cleaved from the resin with 490 ml of a mixture of TFA/DCM (2%). The TFA is extracted with water (490 ml) and the peptide is precipitated, after condensation, with addition of Hexane (1400 ml). Final Yield (59 g, 83%)

Chlorotrityl protection of Fmoc-TGF(38-50)-OH: 65 g of the protected peptide are dissolved with 1300 ml DCM and 16 g of 2-chlorotrityl chloride and 17.5 ml DIPEA are added. The reaction is left to stand for 2 h and monitored by HPLC. DIPEA is extracted from the dichloromethane mixture with 1300 ml of 0.1N hydrochloride acid and Fmoc-TGF(38-50)-OClt is precipitated after condensation with 1400 ml of Hexane and dried under vacuum. Fmoc cleavage is performed with 6 mmol excess of piperidine in NMP (15 ml). 480 ml DCM is added in the reaction mixture and piperidine is extracted from the dichloromethane solution with 480 ml of 0.01N hydrochloride acid. H-TGF(38-50)-OClt is precipitated after condensation with 1400 ml of Hexane, washed 6 times with 400 ml diethylether and dried under vacuum. Final yield 56 g (95%)

Synthesis of Fmoc-TGF(20-37)-OH: 70 g of 2-Chlorotrityl chloride resin are swelled with 350 ml DCM. 48 ml DIPEA and 8.4 g Fmoc-Gly-OH are added. The reaction is left to stand for 3 hours and 21 ml of methanol is added. The resin is filtered and neutralized with 250 ml of DCM/MeOH/DIPEA. The Fmoc-cleavage is performed with the addition of a mixture of 230 ml piperidine/NMP (15%). All couplings were performed with 2.5 mmol excess of Fmoc-amino acids/HOBt/DIC (1:1.2:1.1) in NMP (0.5M). The protected peptide is cleaved from the resin with 490 ml of a mixture of TFA/DCM (2%). The TFA is extracted with water (490 ml) and the peptide is precipitated, after condensation, with addition of Hexane (1400 ml). Final Yield (69 g, 76%)

Synthesis of Boc-TGF(1-19)-OH: 70 g of 2-Chlorotrityl chloride resin are swelled with 350 ml DCM. 48 ml DIPEA and 8.4 g Fmoc-Gly-OH are added. The reaction is left to stand for 3 hours and 21 ml of methanol is added. The resin is filtered and neutralized with 250 ml of DCM/MeOH/DIPEA. The Fmoc-cleavage is performed with the addition of a mixture of 230 ml piperidine/NMP (15%). All couplings were performed with 2.5 mmol excess of Fmoc-amino acids/HOBt/DIC (1:1.2:1.1) in NMP (0.5M). The protected peptide is cleaved from the resin with 490 ml of a mixture of TFA/DCM (2%). The TFA is extracted with water (490 ml) and the peptide is precipitated, after condensation, with addition of Hexane (2000 ml). Final Yield (83 g, 80%)

Synthesis of protected TGF (1-50): 65 g Fmoc-TGF(20-37)-OH is activated with HOBt.H₂O (2.5 g) in 120 ml NMP and EDAC.HCl (2.83 g) and H-TGF(38-50)-OClt (37 g) is added in the activated protected peptide. The completion of the reaction is monitored by HPLC. When Fmoc-TGF(20-37)-OH is <0.5% by area comparing to the corresponding Fmoc-TGF(20-50)-OClt, 8 ml of piperidine is added for the Fmoc-cleavage. 360 ml DCM is added in the reaction mixture and piperidine is extracted from the dichloromethane solution with 360 ml of 0.01N hydrochloride acid. H-TGF(20-50)-OClt is precipitated after condensation with 1700 ml of Hexane, washed 6 times with 420 ml diethylether and dried under vacuum. Final yield 80.2 g (95%)

In 78 g of protected H-TGF(20-50)-OClt dissolved in 400 ml NMP, the activated Boc-TGF(1-19)-OH (55 g), HOBt.H₂O (2.4 g), EDAC.HCl (2.73 g) dissolved in 280 ml NMP is added. The reaction is monitored by HPLC. When H-TGF(20-50)-OClt is <1% by area comparing to crude EGF the reaction is stopped by the addition of 3.2 Lt of water and filtered. The final protected peptide is washed 3 times with water (800 ml) and dried under vacuum until the water content is <3%. Final Yield 121 g (98%)

Iodine oxidation of protected TGF (1-50): The protected peptide (110 g) is dissolved in 1000 ml DCM and an iodine solution (5.4g) in 600 ml 1% TFA/DCM is added. The reaction is left to stand for 1 h and an aqueous solution (1.6 Lt) of sodium sulfate pentahydrate (9.6 g) is added. The DCM/peptide solution is extracted two more times with 1.6 Lt of water, concentrated under vacuum, precipitated with Hexane (1.2 Lt) and washed 3 times with diethylether (440 ml). Final Yield 86 g (97%)

Global Deprotection: 80 g of Boc-TGF(1-50)-OClt is deprotected by its protecting groups by the addition of 5.3 Lt of a mixture consisting of TFA/H₂O/DTT (94:3:3) in RT for 2.5 h. The DTT functions as a scavenger and to avoid possible premature oxidation. The reaction is concentrated under vacuum and crude linear EGF is precipitated by the addition of 1000 ml diethylether and washed three times with diethylether (250 ml). Final yield 48.6 g (97%)

DMSO/DTT oxidation: Crude linear TGF (45 g) is dissolved in 6 Lt DMSO and a water solution (24 Lt) containing Tris (220 g) and Guanidine hydrochloride (280 g) is added in RT. The guanidine hydrochloride functions as a chaotrope. The reaction is left to stand for 24 h and its completion is monitored by HPLC. Final Yield 22%

HPLC Purification: After acidification with 0.2% TFA the crude linear TGF solution is directly loaded on a preparative HPLC column packed with Kromasil C-18, 100A, 13 μm. Crude native h-TGF is purified with a two-step purification process, the first with TFA and the second with ammonium acetate (pH 6). Acetonitrile is used as the organic modifier. Fractions containing native TGF with a purity >97% are further lyophilized. Final Yield 2.1 g (78%)

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

1. A process for preparing an epidermal growth factor-like peptide (EGF-like peptide) comprising the following amino acid sequence,
[SEQ ID NO: 49]
C¹(X)₇-C²(X)₄₋₅C³(X)₁₀₋₁₃C⁴(X)C⁵(X)₈C⁶
wherein C¹-C⁶ are each cysteine and each X is independently a natural or unnatural amino acid, and wherein said EGF-like peptide has three intramolecular disulfide bonds;
said process comprising the steps of:
(I) preparing a first peptide fragment wherein the N-terminal amino acid is protected by a protecting group PG₁, which is selected from Boc and Fmoc;
(II) preparing a second peptide fragment wherein the C-terminal amino acid is protected by a protecting group PG₂, which is selected from trityl, chlorotrityl and t-butyl;
and wherein the amino acid side chains in said first and second peptide fragments are optionally protected;
(III) coupling the C-terminal amino acid of said first peptide fragment with the N-terminal amino acid of said second peptide fragment in solution to form a linear protected EGF-like peptide;
(IV)(a)
(i) treating the linear protected EGF-like peptide formed in step (Ill) with iodine to form an oxidized mixture;
(ii) globally deprotecting the oxidized mixture obtained in step (IV)(a)(i) by treating with trifluoroacetic acid (TFA);
(iii) treating the deprotected oxidized mixture obtained in step (IV)(a)(ii) with DMSO/DTT to form a crude EGF-like peptide; or
(IV)(b)
(i) globally deprotecting the linear protected EGF-like peptide obtained in step (III) by treating with trifluoroacetic acid (TFA);
(ii) treating the deprotected mixture obtained in step (IV)(b)(i) with DMSO to form a crude EGF-like peptide; and
(V) optionally purifying the crude EGF-like peptide.

2. A process according to claim 1 wherein the first peptide fragment is prepared by coupling two or more peptide sub-fragments.

3. A process according to claim 1 wherein the first peptide fragment is prepared by solid phase peptide synthesis.

4. A process according to any preceding claim wherein the second peptide fragment is prepared by coupling two or more peptide sub-fragments.

5. A process according to any one of claims 1 to 3 wherein the second peptide fragment is prepared by solid phase peptide synthesis.

6. A process according claim 1 wherein the EGF-like peptide is EGF, or an analogue or variant thereof.

7. A process according claim 1 wherein the EGF-like peptide is murine EGF, or an analogue or variant thereof.

8. A process according claim 1 wherein EGF-like peptide is human EGF, or an analogue or variant thereof.

9. A process according to claim 1 wherein the C-terminal amino acid of the first peptide fragment is glycine.

10. A process according to claim 1 wherein the EGF-like peptide comprises the following sequence:
[SEQ ID NO: 1]
H-Asn¹-Ser²-Asp³-Ser⁴-Glu⁵-Cys⁶-Pro⁷-Leu⁸-Ser⁹-His¹⁰-
Asp¹¹-Gly¹²-Tyr¹³-Cys¹⁴-Leu¹⁵-His¹⁶-Asp¹⁷-Gly¹⁸-Val¹⁹-
Cys²⁰-Met²¹-Tyr²²-Ile²³-Glu²⁴-Ala²⁵-Leu²⁶-Asp²⁷-Lys²⁸-
Tyr²⁹-Ala³⁰-Cys³¹-Asn³²-Cys³³-Val³⁴-Val³⁵-Gly³⁶-Tyr³⁷-
Ile³⁸-Gly³⁹-Glu⁴⁰-Arg⁴¹-Cys⁴²-Gln⁴³-Tyr⁴⁴-Arg⁴⁵-Asp⁴⁶-
Leu⁴⁷-Lys⁴⁸-Trp⁴⁹-Trp⁵⁰-Glu⁵¹-Leu⁵²-Arg⁵³-OH
or a variant thereof,
and said process comprises:
coupling a first peptide fragment comprising the sequence:
PG₁-Asn¹-Ser²-Asp³-Ser⁴-Glu⁵-Cys⁶-Pro⁷-Leu⁸-Ser⁹-His¹⁰-Asp¹¹-Gly¹²-Tyr¹³-Cys¹⁴-Leu¹⁵-His¹⁶-Asp¹⁷-Gly¹⁸-OH [SEQ ID NO: 2] or a variant thereof,
wherein:
PG₁ is an N-terminal protecting group selected from Boc and Fmoc; and
the C-terminal amino acid is optionally in the form of an activated carboxylic acid derivative;
in solution with a second peptide fragment comprising the sequence:
H-Val¹⁹-Cys²⁰-Met²¹-Tyr²²-Ile²³-Glu²⁴-Ala²⁵-Leu²⁶-Asp²⁷-Lys²⁸-Tyr²⁹-Ala³⁰-Cys³¹-Asn³²-Cys³³-Val³⁴-Val³⁵-Gly³⁶-Tyr³⁷-Ile³⁸-Gly³⁹-Glu⁴⁰-Arg⁴¹-Cys⁴²-Gln⁴³-Tyr⁴⁴-Arg⁴⁵-Asp⁴⁶-Leu⁴⁷-Lys⁴⁸-Trp⁴⁹-Trp⁵⁰-Glu⁵¹-Leu⁵²-Arg⁵³-O-PG₂ [SEQ ID NO: 3] or a variant thereof,
wherein PG₂ is a protecting group selected from chlorotrityl and t-butyl;
and wherein one or more of the amino acid residues in said first and second peptide fragments is optionally protected, preferably with an acid-cleavable protecting group.

11. A process according to claim 10 wherein the EGF-like peptide comprises the following sequence:
H-Asn¹(P)-Ser²(P)-Asp³(P)-ψSer⁴-Glu⁵(P)-Cys⁶(P)-Pro⁷-Leu⁸-4)Ser⁹-His¹⁰(P)-Asp¹¹(P)-Gly¹²-Tyr¹³(P)-Cys¹⁴(P)-Leu¹⁵-His¹⁶(P)-Asp¹⁷(P)-Gly¹⁸-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-OH [SEQ ID NO: 9] or a variant thereof,
wherein the first peptide fragment comprises the following sequence:
PG₁-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-Tyr¹³(P)-Cys¹⁴(P)-Leu¹⁵-His¹⁶(P)-Asp¹⁷(P)-Gly¹⁸-OH [SEQ ID NO: 10] or a variant thereof;
and the second peptide fragment comprises the following sequence:
H-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 11] or a variant thereof;
wherein each P represents a side chain protecting group which may be the same or different.

12. A process according to claim 10 wherein the EGF-like peptide comprises the following sequence:
H-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-Tyr¹³(tBu)-Cys¹⁴(Trt)-Leu¹⁵-His¹⁶(Trt)-Asp¹⁷(tBu)-Gly¹⁸-Val¹⁹-Cys²⁰Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-OH [SEQ ID NO: 12] or a variant thereof;
and wherein the first peptide fragment comprises the following sequence:
PG₁-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-Tyr¹³(tBu)-Cys¹⁴(Trt)-Leu¹⁵-His¹⁶(Trt)-Asp¹⁷(tBu)-Gly¹⁸-OH [SEQ ID NO: 13] or a variant thereof.
and the second peptide fragment comprises the following sequence:
H-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 14] or a variant thereof.

13. A process according to claim 11 or claim 12 wherein the first peptide fragment comprising the sequence PG₁-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-Tyr¹³(P)-Cys¹⁴(P)-Leu¹⁵-His¹⁶(P)-Asp¹⁷(P)-Gly¹⁸-OH [SEQ ID NO: 10] or PG₁-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-Tyr¹³(tBu)-Cys¹⁴(Trt)-Leu¹⁵-His¹⁶(Trt)-Asp¹⁷(tBu)-Gly¹⁸-OH [SEQ ID NO: 13] is prepared by solid phase synthesis starting from Fmoc-Gly-OH.

14. A process according to claim 11 wherein the first peptide fragment comprising the sequence PG₁-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-Tyr¹³(P)-Cys¹⁴(P)-Leu¹⁵-His¹⁶(P)-Asp¹⁷(P)-Gly¹⁸-OH [SEQ ID NO: 15] is prepared by fragment condensation of PG₁-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-OH [SEQ ID NO: 20] and H-Tyr¹³(P)-Cys¹⁴(P)-Leu¹⁵-His¹⁶(P)-Asp¹⁷(P)-Gly¹⁸-O-PG₂ [SEQ ID NO: 17], followed by removal of protecting group PG₂.

15. A process according to claim 12 wherein the first peptide fragment comprising the sequence PG₁-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-Tyr¹³(tBu)-Cys¹⁴(Trt)-Leu¹⁵-His¹⁶(Trt)-Asp¹⁷(tBu)-Gly¹⁸-OH [SEQ ID NO: 13] is prepared by fragment condensation of PG₁-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-OH [SEQ ID NO: 18] and H-Tyr¹³(tBu)-Cys¹⁴(Trt)-Leu¹⁵-His¹⁶(Trt)-Asp¹⁷(tBu)-Gly¹⁸-O-PG₂ [SEQ ID NO: 19], followed by removal of protecting group PG₂.

16. A process according to claim 14 or claim 15 wherein the peptide fragment comprising the sequence PG₁-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-OH [SEQ ID NO: 20] or PG₁-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-OH [SEQ ID NO: 21] is prepared by solid phase synthesis starting from Fmoc-Gly-OH.

17. A process according to claim 14 or claim 15 wherein the peptide fragment comprising the sequence H-Tyr¹³(P)-Cys¹⁴(P)-Leu¹⁵-His¹⁶(P)-Asp¹⁷(P)-Gly¹⁸-O-PG₂ [SEQ ID NO: 17] or H-Tyr¹³(tBu)-Cys¹⁴(Trt)-Leu¹⁵-His¹⁶(Trt)-Asp¹⁷(tBu)-Gly¹⁸-O-PG₂ [SEQ ID NO: 19] is prepared by solid phase synthesis starting from Fmoc-Gly-OH.

18. A process according to claim 11 wherein the second peptide fragment comprising the sequence H-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 11] is prepared by fragment condensation of PG₁-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-OH [SEQ ID NO: 22] and H-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 23], followed by removal of protecting group PG₁.

19. A process according to claim 12 wherein the second peptide fragment comprising the sequence H-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 14] is prepared by fragment condensation of PG₁-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-OH [SEQ ID NO: 24] and H-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 25], followed by removal of protecting group PG 1 .

20. A process according to claim 11 wherein the second peptide fragment comprising the sequence H-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 11] is prepared by fragment condensation of PG₁-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-OH [SEQ ID NO: 26] and H-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 27], followed by removal of protecting group PG₁.

21. A process according to claim 12 wherein the second peptide fragment comprising the sequence H-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 14] is prepared by fragment condensation of PG₁-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-OH [SEQ ID NO: 28] and H-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 29], followed by removal of protecting group PG₁.

22. A process according to claim 18 wherein the peptide fragment comprising the sequence H-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P) -Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 23] is prepared by fragment condensation of PG₁-Tyr³⁷(P)-Ile³⁸-Gly³⁹-OH [SEQ ID NO: 30] and H-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 27], followed by removal of protecting group PG₁.

23. A process according to claim 19 wherein the peptide fragment comprising the sequence H-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 25] is prepared by fragment condensation of PG₁-Tyr³⁷(tBu)-Ile³⁸-Gly³⁹-OH [SEQ ID NO: 31] and H-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 29], followed by removal of protecting group PG₁.

24. A process according to claim 18 or claim 19 wherein the peptide fragment PG₁-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-OH [SEQ ID NO: 22] or PG 1-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-OH [SEQ ID NO: 24] is prepared by solid phase peptide synthesis starting with Fmoc-Gly-OH.

25. A process according to claim 18 or claim 19 wherein the peptide fragment H-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 23] or H-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 25] is prepared by solid phase peptide synthesis starting with Fmoc-Arg(P) or Fmoc-Arg(Pbf).

26. A process according to claim 20 or claim 21 wherein the peptide fragment PG₁-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-OH [SEQ ID NO: 26] or PG₁-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-OH [SEQ ID NO: 28] is prepared by solid phase peptide synthesis starting with Fmoc-Gly-OH.

27. A process according to claim 20 or claim 21 wherein the peptide fragment H-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 27] or H-Glu(tBu)m-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 29] is prepared by solid phase peptide synthesis using Fmoc-Arg(P) or Fmoc-Arg(Pbf).

28. A process according to claim 1 wherein the EGF-like peptide comprises the following sequence:
[SEQ ID NO: 1]
H-Asn¹-Ser²-Asp³-Ser⁴-Glu⁵-Cys⁶-Pro⁷-Leu⁸-Ser⁹-His¹⁰-
Asp¹¹-Gly¹²-Tyr¹³-Cys¹⁴-Leu¹⁵-His¹⁶-Asp¹⁷-Gly¹⁸-Val¹⁹-
Cys²⁰-Met²¹-Tyr²²-Ile²³-Glu²⁴-Ala²⁵-Leu²⁶-Asp²⁷-Lys²⁸-
Tyr²⁹-Ala³⁰-Cys³¹-Asn³²-Cys³³-Val³⁴-Val³⁵-Gly³⁶-Tyr³⁷-
Ile³⁸-Gly³⁹-Glu⁴⁰-Arg⁴¹-Cys⁴²-Gln⁴³-Tyr⁴⁴-Arg⁴⁵-Asp⁴⁶-
Leu⁴⁷-Lys⁴⁸-Trp⁴⁹-Trp⁵⁰-Glu⁵¹-Leu⁵²-Arg⁵³-OH
or a variant tnereot,
and said process comprises:
coupling a first peptide fragment comprising the sequence:
PG₁-Asn¹-Ser²-Asp³-Ser⁴-Glu⁵-Cys⁶-Pro⁷-Leu⁸-Ser⁹-His¹⁰-Asp¹¹-Gly¹²-OH [SEQ ID NO: 4] or a variant thereof;
wherein:
PG₁ is an N-terminal protecting group selected from Boc and Fmoc; and
the C-terminal amino acid is optionally in the form of an activated carboxylic acid derivative;
in solution with a second peptide fragment comprising the sequence:
H-Tyr¹³-Cys¹⁴-Leu¹⁵-His¹⁶-Asp¹⁷-Gly¹⁸-Val¹⁹-Cys²⁰-Met²¹-Tyr²²-Ile²³-Glu²⁴-Ala²⁵-Leu²⁶-AsP²⁷-Lys²⁸-Tyr²⁹-Ala³⁰-Cys³¹-Asn³²-Cys³³-Val³⁴-Val³⁵-Gly³⁶-Tyr³⁷-Ile³⁸-Gly³⁹-Glu⁴⁰-Arg⁴¹-Cys⁴²-Gln⁴³-Tyr⁴⁴-Arg⁴⁵-Asp⁴⁶-Leu⁴⁷-Lys⁴⁸-Trp⁴⁹-Trp⁵⁰-Glu⁵¹-Leu⁵²-Arg⁵³-O-PG₂ [SEQ ID NO: 5] or a variant thereof;
wherein PG₂ is a protecting group selected from chlorotrityl and t-butyl;
and wherein one or more of the amino acid residues in said first and second peptide fragments is optionally protected, preferably with an acid-cleavable protecting group.

29. A process according to claim 28 wherein the EGF-like peptide comprises the following sequence:
H-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-Tyr¹³(P)-Cys¹⁴(P)-Leu¹⁵-His¹⁶(P)-Asp¹⁷(P)-Gly¹⁸-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-OH [SEQ ID NO: 9] or a variant thereof,
wherein the first peptide fragment comprises the following sequence:
PG₁-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-OH [SEQ ID NO: 34] or a variant thereof;
and the second peptide fragment comprises the following sequence:
H-Tyr¹³(P)-Cys¹⁴(P)-Leu¹⁵-His¹⁶(P)-Asp¹⁷(P)-Gly¹⁸-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 32] or a variant thereof;
wherein each P represents a side chain protecting group which may be the same or different.

30. A process according to claim 28 wherein the EGF-like peptide comprises the following sequence:
H-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-Tyr¹³(tBu)-Cys¹⁴(Trt)-Leu¹⁵-His¹⁶(Trt)-Asp¹⁷(tBu)-Gly¹⁸-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-OH [SEQ ID NO: 12] or a variant thereof;
and wherein the first peptide fragment comprises the following sequence:
PG₁-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-OH [SEQ ID NO: 18] or a variant thereof, and the second peptide fragment comprises the following sequence:
H-Tyr¹³(tBu)-Cys¹⁴(Trt)-Leu¹⁵-His¹⁶(Trt)-Asp¹⁷(tBu)-Gly¹⁸-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 33] or a variant thereof.

31. A process according to claim 29 or claim 30 wherein the first peptide fragment PG₁-Asn¹(P)-Ser²(P)-Asp³(P)-ψSer⁴-Glu⁵(P)-Cys⁶(P)-Pro⁷-Leu⁸-ψSer⁹-His¹⁰(P)-Asp¹¹(P)-Gly¹²-OH [SEQ ID NO: 34] or PG₁-Asn¹(Trt)-Ser²(tBu)-Asp³(tBu)-ψSer⁴-Glu⁵(tBu)-Cys⁶(Trt)-Pro⁷-Leu⁸-ψSer⁹-His¹⁰(Trt)-Asp¹¹(tBu)-Gly¹²-OH [SEQ ID NO: 18] is prepared by solid phase peptide synthesis starting with Fmoc-Gly-OH.

32. A process according to claim 29 or claim 30 wherein the second peptide fragment H-Tyr¹³(P)-Cys¹⁴(P)-Leu¹⁵-His¹⁶(P)-Asp¹⁷(P)-Gly¹⁸-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 32] or H-Tyr¹³(tBu)-Cys¹⁴(Trt)-Leu¹⁵-His¹⁶(Trt)-Asp¹⁷(tBu)-Gly¹⁸-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 33] is prepared by solid phase peptide synthesis starting with Fmoc-Arg(P) or Fmoc-Arg(Pbf).

33. A process according to claim 29 wherein the second peptide fragment H-Tyr¹³(P)-Cys¹⁴(P)-Leu¹⁵-His¹⁶(P)-Asp¹⁷(P)-Gly¹⁸-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 32] is prepared by fragment condensation of PG₁-Tyr¹³(P)-Cys¹⁴(P)-Leu¹⁵-His¹⁶(P)-Asp¹⁷(P)-Gly¹⁸-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-OH [SEQ ID NO: 35] and H-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 23], followed by removal of protecting group PG₁.

34. A process according to claim 30 wherein the second peptide fragment or H-Tyr¹³(tBu)-Cys¹⁴(Trt)-Leu¹⁵-His¹⁶(Trt)-Asp¹⁷(tBu)-Gly¹⁸-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 33] is prepared by fragment condensation of PG₁-Tyr¹³(tBu)-Cys¹⁴(Trt)-Leu¹⁵-His¹⁶(Trt)-Asp¹⁷(tBu)-Gly¹⁸-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-OH [SEQ ID NO: 36] and H-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 25], followed by removal of protecting group PG₁.

35. A process according to claim 33 or claim 34 wherein the peptide fragment PG₁-Tyr¹³(P)-Cys¹⁴(P)-Leu¹⁵-His¹⁶(P)-Asp¹⁷(P)-Gly¹⁸-Val¹⁹-Cys²⁰(P)-Met²¹-Tyr²²(P)-Ile²³-Glu²⁴(P)-Ala²⁵-Leu²⁶-Asp²⁷(P)-Lys²⁸(P)-Tyr²⁹(P)-Ala³⁰-Cys³¹(P)-Asn³²(P)-Cys³³(P)-Val³⁴-Val³⁵-Gly³⁶-OH [SEQ ID NO: 35] or PG₁-Tyr¹³(tBu)-Cys¹⁴(Trt)-Leu¹⁵-His¹⁶(Trt)-Asp¹⁷(tBu)-Gly¹⁸-Val¹⁹-Cys²⁰(Trt)-Met²¹-Tyr²²(tBu)-Ile²³-Glu²⁴(tBu)-Ala²⁵-Leu²⁶-Asp²⁷(tBu)-Lys²⁸(Boc)-Tyr²⁹(tBu)-Ala³⁰-Cys³¹(Trt)-Asn³²(Trt)-Cys³³(Trt)-Val³⁴-Val³⁵-Gly³⁶-OH [SEQ ID NO: 36] is prepared by solid phase peptide synthesis starting with Fmoc-Gly-OH.

36. A process according to claim 33 or claim 34 wherein the peptide fragment H-Tyr³⁷(P)-Ile³⁸-Gly³⁹-Glu⁴⁰(P)-Arg⁴¹(P)-Cys⁴²(P)-Gln⁴³(P)-Tyr⁴⁴(P)-Arg⁴⁵(P)-Asp⁴⁶(P)-Leu⁴⁷-Lys⁴⁸(P)-Trp⁴⁹(P)-Trp⁵⁰(P)-Glu⁵¹(P)-Leu⁵²-Arg⁵³(P)-O-PG₂ [SEQ ID NO: 23] or H-Tyr³⁷(tBu)-Ile³⁸-Gly³⁹-Glu(tBu)⁴⁰-Arg⁴¹(Pbf)-Cys⁴²(Trt)-Gln⁴³(Trt)-Tyr⁴⁴(tBu)-Arg⁴⁵(Pbf)-Asp⁴⁶(tBu)-Leu⁴⁷-Lys⁴⁸(Boc)-Trp⁴⁹(Boc)-Trp⁵⁰(Boc)-Glu⁵¹(tBu)-Leu⁵²-Arg⁵³(Pbf)-O-PG₂ [SEQ ID NO: 25] is prepared by solid phase peptide synthesis starting with Fmoc-Arg(P) or Fmoc-Arg(Pbf).

37. A process according claim 1 wherein the EGF-like peptide is transforming growth factor-α (TGF-α), or an analogue or variant thereof.

38. A process according claim 37 wherein the EGF-like peptide is human transforming growth factor-α (hTGF-α), or an analogue or variant thereof.

39. A process according to claim 37 wherein the EGF-like peptide comprises the following sequence:
H-Val¹-Val²-Ser³-His⁴-Phe⁵-Asn⁶-Asp⁷-Cys⁸-Pro⁹-Asp¹⁰-Ser¹¹-His¹²-Thr¹³-Gln¹⁴-Phe¹⁵-Cys¹⁶-Phe¹⁷-His¹⁸-Gly¹⁹-Thr²⁰-Cys²¹-Arg²²-Phe²³-Leu²⁴-Val²⁵-Gln²⁶-Glu²⁷-Asp²⁸-Lys²⁹-Pro³⁰-Ala³¹-Cys³²-Val³³-Cys³⁴-His³⁵-Ser³⁶-Gly³⁷-Tyr³⁸-Val³⁹-Gly⁴⁰-Ala⁴¹-Arg⁴²-Cys⁴³-Glu⁴⁴-His⁴⁵-Ala⁴⁶-Asp⁴⁷-Leu⁴⁸-Leu⁴⁹-Ala⁵⁰-OH [SEQ ID NO: 6] or a variant thereof;
and said process comprises:
coupling a first peptide fragment comprising the sequence:
PG₁-Val¹-Val²-Ser³-His⁴-Phe⁵-Asn⁶-Asp⁷-Cys⁸-Pro⁹-Asp¹⁰-Ser¹¹-His¹²-Thr¹³-Gln¹⁴-Phe¹⁵-Cys¹⁶-Phe¹⁷-His¹⁸-Gly¹⁹-OH [SEQ ID NO: 7] or a variant thereof;
wherein:
PG₁ is an N-terminal protecting group selected from Boc and Fmoc; and
the C-terminal amino acid is optionally in the form of an activated carboxylic acid derivative;
in solution with a second peptide fragment comprising the sequence:
H-Thr²⁰-Cys²¹-Arg²²-Phe²³-Leu²⁴-Val²⁵-Gln²⁶-Glu²⁷-Asp²⁸-Lys²⁹-Pro³⁰° -Ala³¹-Cys³²-Val³³-Cys³⁴-His³⁵-Ser³⁶-Gly³⁷-Tyr³⁸-Val³⁹-Gly⁴⁰-Ala⁴¹-Arg⁴²-Cys⁴³-Glu⁴⁴-His⁴⁵-Ala⁴⁶-Asp⁴⁷-Leu⁴⁸-Leu⁴⁹-Ala⁵⁰-O-PG₂ [SEQ ID NO: 8] or a variant thereof,
wherein PG₂ is a protecting group selected from chlorotrityl and t-butyl;
and wherein one or more of the amino acid residues in said first and second peptide fragments is optionally protected, preferably with an acid-cleavable protecting group.

40. A process according to claim 37 wherein the EGF-like peptide comprises the following sequence:
H-Val¹-Val²-Ser³(P)-His⁴(P)-Phe⁵-Asn⁶(P)-Asp⁷(P)-Cys⁸(P)-Pro⁹-Asp¹⁰(P)-ψSer¹¹-His¹²(P)-Thr¹³(P)-Gln¹⁴(P)-Phe¹⁵-Cys¹⁶(P)-Phe¹⁷-His¹⁸(P)-Gly¹⁹-Thr²⁰(P)-Cys²¹(P)-Arg²²(P)-Phe²³-Leu²⁴-Val²⁵-Gln²⁶-Glu²⁷(P)-Asp²⁸(P)-Lys²⁹(P)-Pro³⁰-Ala³¹-Cys³²(P)-Val³³-Cys³⁴(P)-His³⁵-Ser³⁶(P)-Gly³⁷-Tyr³⁸(P)-Val³⁹-Gly⁴⁰-Ala⁴¹-Arg⁴²(P)-Cys⁴³(P)-Glu⁴⁴(P)-His⁴⁵(P)-Ala⁴⁶-Asp⁴⁷(P)-Leu⁴⁸-Leu⁴⁹-Ala⁵⁰-OH [SEQ ID NO: 50] or a variant thereof,
wherein the first peptide fragment comprises the following sequence:
PG₁-Val¹-Val²-Ser³(P)-His⁴(P)-Phe⁵-Asn⁶(P)-Asp⁷(P)-Cys⁸(P)-Pro⁹-Asp¹⁰(P)-ψSer¹¹-His¹²(P)-Thr¹³(P)-Gln¹⁴(P)-Phe¹⁵-Cys¹⁶(P)-Phe¹⁷-His¹⁸(P)-Gly¹⁹-OH [SEQ ID NO: 38] or a variant thereof,
and the second peptide fragment comprises the following sequence:
H-Thr²⁰(P)-Cys²¹(P)-Arg²²(P)-Phe²³-Leu²⁴-Val²⁵-Gln²⁶-Glu²⁷(P)-Asp²⁸(P)-Lys²⁹(P)-Pro³⁰-Ala³¹-Cys³²(P)-Val³³-Cys³⁴(P)-His³⁵-Ser³⁶(P)-Gly³⁷-Tyr³⁸(P)-Val³⁹-Gly⁴⁰-Ala⁴¹-Arg⁴²(P)-Cys⁴³(P)-Glu⁴⁴(P)-His⁴⁵(P)-Ala⁴⁶-Asp⁴⁷(P)-Leu⁴⁸-Leu⁴⁹-Ala⁵⁰-O-PG₂ [SEQ ID NO: 39] or a variant thereof,
wherein each P represents a side chain protecting group which may be the same or different.

41. A process according to claim 37 wherein the EGF-like peptide comprises the following sequence:
H-Val¹-Val²-Ser³(tBu)-His⁴(Trt)-Phe⁵-Asn⁶(Trt)-Asp⁷(tBu)-Cys⁸(Trt)-Pro⁹-Asp¹⁰(tBu)-ψSer¹¹-His¹²(Trt)-Thr¹³(tBu)-Gln¹⁴(Trt)-Phe¹⁵-Cys¹⁶(Trt)-Phe¹⁷-His¹⁸(Trt)-Gly¹⁹-Thr²⁰(tBu)-Cys²¹(Trt)-Arg²²(Pbf)-Phe²³-Leu²⁴-Val²⁵-Gln²⁶-Glu²⁷(tBu)-Asp²⁸(tBu)-Lys²⁹(Boc)-Pro³⁰-Ala³¹-Cys³²(Trt)-Val³³-Cys³⁴(Trt)-His³⁵-Ser³⁶(tBu)-Gly³⁷-Tyr³⁸(tBu)-Val³⁹-Gly⁴⁰-Ala⁴¹-Arg⁴²(Pbf)-Cys⁴³(Trt)-Glu⁴⁴(tBu)-His⁴⁵(Trt)-Ala⁴⁶-Asp⁴⁷(tBu)-Leu⁴⁸-Leu⁴⁹-Ala⁵⁰-OH [SEQ ID NO: 51] or a variant thereof,
wherein the first peptide fragment comprises the following sequence:
PG₁-Val¹-Val²-Ser³(tBu)-His⁴(Trt)-Phe⁵-Asn⁶(Trt)-Asp⁷(tBu)-Cys⁸(Trt)-Pro⁹-Asp¹⁰(tBu)-ψSer¹¹-His¹²(Trt)-Thr¹³(tBu)-Gln¹⁴(Trt)-Phe¹⁵-Cys¹⁶(Trt)-Phe¹⁷-His¹⁸(Trt)-Gly¹⁹-OH [SEQ ID NO: 41] or a variant thereof,
and the second peptide fragment comprises the following sequence:
H-Thr²⁰(tBu)-Cys²¹(Trt)-Arg²²(Pbf)-Phe²³-Leu²⁴-Val²⁵-Gln²⁶-Glu²⁷(tBu)-Asp²⁸(tBu)-Lys²⁹(Boc)-Pro³⁰-Ala³¹-Cys³²(Trt)-Val³³-Cys³⁴(Trt)-His³⁵-Ser³⁶(tBu)-Gly³⁷-Tyr³⁸(tBu)-Val³⁹-Gly⁴⁰-Ala⁴¹-Arg⁴²(Pbf)-Cys⁴³(Trt)-Glu⁴⁴(tBu)-His⁴⁵(Trt)-Ala⁴⁶-Asp⁴⁷(tBu)-Leu⁴⁸-Leu⁴⁹-Ala⁵⁰-O-PG₂ [SEQ ID NO: 42] or a variant thereof.

42. A process according to claim 40 or claim 41 wherein the first peptide fragment PG₁-Val¹-Val²-Ser³(P)-His⁴(P)-Phe⁵-Asn⁶(P)-Asp⁷(P)-Cys⁸(P)-Pro⁹-Asp¹⁰(P)-ψSer¹¹-His¹²(P)-Thr¹³(P)-Gln¹⁴(P)-Phe¹⁵-Cys¹⁶(P)-Phe¹⁷-His¹⁸(P)-Gly¹⁹-OH [SEQ ID NO: 38] or PG₁-Val¹-Val²-Ser³(tBu)-His⁴(Trt)-Phe⁵-Asn⁶(Trt)-Asp⁷(tBu)-Cys⁸(Trt)-Pro⁹-Asp¹⁰(tBu)-ψSer¹¹-His¹²(Trt)-Thr¹³(tBu)-Gln¹⁴(Trt)-Phe¹⁵-Cys¹⁶(Trt)-Phe¹⁷-His¹⁸(Trt)-Gly¹⁹-OH [SEQ ID NO: 41] is prepared by solid phase peptide synthesis starting with Fmoc-Gly-OH.

43. A process according to claim 40 wherein the second peptide fragment comprising the sequence H-Thr²⁰(P)-Cys²¹(P)-Arg²²(P)-Phe²³-Leu²⁴-Val²⁵-Gln²⁶-Glu²⁷(P)-Asp²⁸(P)-Lys²⁹(P)-Pro³⁰-Ala³¹-Cys³²(P)-Val³³-Cys³⁴(P)-His³⁵-Ser³⁶(P)-Gly³⁷-Tyr³⁸(P)-Val³⁹-Gly⁴⁰-Ala⁴¹-Arg⁴²(P)-Cys⁴³(P)-Glu⁴⁴(P)-His⁴⁵(P)-Ala⁴⁶-Asp⁴⁷(P)-Leu⁴⁸-Leu⁴⁹-Ala⁵⁰-O-PG₂ [SEQ ID NO: 39] is prepared by fragment condensation of PG₁-Thr²⁰(P)-Cys²¹(P)-Arg²²(P)-Phe²³-Leu²⁴-Val²⁵-Gln²⁶-Glu²⁷(P)-Asp²⁸(P)-Lys²⁹(P)-Pro³⁰-Ala³¹-Cys³²(P)-Val³³-Cys³⁴(P)-His³⁵-Ser³⁶(P)-Gly³⁷-OH [SEQ ID NO: 43] and H-Tyr³⁸(P)-Val³⁹-Gly⁴⁰-Ala⁴¹-Arg⁴²(P)-Cys⁴³(P)-Glu⁴⁴(P)-His⁴⁵(P)-Ala⁴⁶-Asp⁴⁷(P)-Leu⁴⁸-Leu⁴⁹-Ala⁵⁰-O-PG₂ [SEQ ID NO: 44], followed by removal of protecting group PG₁.

44. A process according to claim 41 wherein the second peptide fragment comprising the sequence H-Thr²⁰(tBu)-Cys²¹(Trt)-Arg²²(Pbf)-Phe²³-Leu²⁴-Val²⁵-Gln²⁶-Glu²⁷(tBu)-Asp²⁸(tBu)-Lys²⁹(Boc)-Pro³⁰-Ala³¹-Cys³²(Trt)-Val³³-Cys³⁴(Trt)-His³⁵-Ser³⁶(tBu)-Gly³⁷-Tyr³⁸(tBu)-Val³⁹-Gly⁴⁰-Ala⁴¹-Arg⁴²(Pbf)-Cys⁴³(Trt)-Glu⁴⁴(tBu)-His⁴⁵(Trt)-Ala⁴⁶-Asp⁴⁷(tBu)-Leu⁴⁸-Leu⁴⁹-Ala⁵⁰-O-PG₂ [SEQ ID NO: 45] is prepared by fragment condensation of PG₁-Thr²⁰(tBu)-Cys²¹(Trt)-Arg²²(Pbf)-Phe²³-Leu²⁴-Val²⁵-Gln²⁶-Glu²⁷(tBu)-Asp²⁸(tBu)-Lys²⁹(Boc)-Pro³⁰-Ala³¹-Cys³²(Trt)-Val³³-Cys³⁴(Trt)-His³⁵-Ser³⁶(tBu)-Gly³⁷-OH [SEQ ID NO: 46] and H-Tyr³⁸(tBu)-Val³⁹-Gly⁴⁰-Ala⁴¹-Arg⁴²(Pbf)-Cys⁴³(Trt)-Glu⁴⁴(tBu)-His⁴⁵(Trt)-Ala⁴⁶-Asp⁴⁷(tBu)-Leu⁴⁸-Leu⁴⁹-Ala⁵⁰-O-PG₂ [SEQ ID NO: 47] , followed by removal of protecting group PG₁.

45. A process according to claim 43 or claim 44 wherein the peptide fragment H-Tyr³⁸(P)-Val³⁹-Gly⁴⁰-Ala⁴¹-Arg⁴²(P)-Cys⁴³(P)-Glu⁴⁴(P)-His⁴⁵(P)-Ala⁴⁶-Asp⁴⁷(P)-Leu48- -Leu⁴⁹-Ala⁵⁰-O-PG₂ [SEQ ID NO: 48] or H-Tyr³⁸(tBu)-Val³⁹-Gly⁴⁰-Ala⁴¹-Arg⁴²(Pbf)-Cys⁴³(Trt)-Glu⁴⁴(tBu)-His⁴⁵(Trt)-Ala⁴⁶-Asp⁴⁷(tBu)-Leu⁴⁸-Leu⁴⁹-Ala⁵⁰-O-PG₂ [SEQ ID NO: 47] is prepared by solid phase peptide synthesis starting with Fmoc-Ala-OH.

46. A process according to claim 43 or claim 44 wherein the peptide fragment PG₁-Thr²⁰(P)-Cys²¹(P)-Arg²²(P)-Phe²³-Leu²⁴-Val²⁵-Gln²⁶-Glu²⁷(P)-Asp²⁸(P)-Lys²⁹(P)-Pro³⁰-Ala³¹-Cys³²(P)-Val³³-Cys³⁴(P)-His³⁵-Ser³⁶(P)-Gly³⁷-OH [SEQ ID NO: 43] or PG₁-Thr²⁰(tBu)-Cys²¹(Trt)-Arg²²(Pbf)-Phe²³-Leu²⁴-Val²⁵-Gln²⁶-Glu²⁷(tBu)-Asp²⁸(tBu)-Lys²⁹(Boc)-Pro³⁰-Ala³¹-Cys³²(Trt)-Val³³-Cys³⁴(Trt)-His³⁵-Ser³⁶(tBu)-Gly³⁷-OH [SEQ ID NO: 46] is prepared by solid phase peptide synthesis starting with Fmoc-Gly-OH.

47. A process according to any preceding claim wherein PG₂ is chlorotrityl.

48. A process according to any preceding claim wherein PG₁ is Boc.

49. A process for preparing an EGF-like peptide comprising the following sequence:
[SEQ ID NO: 1]
H-Asn¹-Ser²-Asp³-Ser⁴-Glu⁵-Cys⁶-Pro⁷-Leu⁸-Ser⁹-His¹⁰-
Asp¹¹-Gly¹²-Tyr¹³-Cys¹⁴-Leu¹⁵-His¹⁶-Asp¹⁷-Gly¹⁸-Val¹⁹-
Cys²⁰-Met²¹-Tyr²²-Ile²³-Glu²⁴-Ala²⁵-Leu²⁶-Asp²⁷-Lys²⁸-
Tyr²⁹-Ala³⁰-Cys³¹-Asn³²-Cys³³-Val³⁴-Val³⁵-Gly³⁶-Tyr³⁷-
Ile³⁸-Gly³⁹-Glu⁴⁰-Arg⁴¹-Cys⁴²-Gln⁴³-Tyr⁴⁴-Arg⁴⁵-Asp⁴⁶-
Leu⁴⁷-Lys⁴⁸-Trp⁴⁹-Trp⁵⁰-Glu⁵¹-Leu⁵²-Arg⁵³-OH
or a variant thereof,
wherein said process comprises:
coupling a first peptide fragment comprising the sequence:
PG₁-Asn¹-Ser²-Asp³-Ser⁴-Glu⁵-Cys⁶-Pro⁷-Leu⁸-Ser⁹-His¹⁰-Asp¹¹-Gly¹²-Tyr¹³-Cys¹⁴-Leu¹⁵-His¹⁶-Asp¹⁷-Gly¹⁸-OH [SEQ ID NO: 2] or a variant thereof,
wherein:
PG₁ is an N-terminal protecting group, preferably selected from Boc and Fmoc; and
the C-terminal amino acid is optionally in the form of an activated carboxylic acid derivative;
in solution with a second peptide fragment comprising the sequence:
H-Val¹⁹-Cys²⁰-Met²¹-Tyr²²-Ile²³-Glu²⁴-Ala²⁵-Leu²⁶-Asp²⁷-Lys²⁸-Tyr²⁹-Ala³⁰-Cys³¹-Asn³²-Cys³³-Val³⁴-Val³⁵-Gly³⁶-Tyr³⁷-Ile³⁸-Gly³⁹-Glu⁴⁰-Arg⁴¹-Cys⁴²-Gln⁴³-Tyr⁴⁴-Arg⁴⁵-Asp⁴⁶-Leu⁴⁷-Lys⁴⁸-Trp⁴⁹-Trp⁵⁰-Glu⁵¹-Leu⁵²-Arg⁵³-O-PG₂ [SEQ ID NO: 3] or a variant thereof,
wherein PG₂ is a protecting group, preferably selected from chlorotrityl and t-butyl;
and wherein one or more of the amino acid residues in said first and second peptide fragments is optionally protected, preferably with an acid-cleavable protecting group; and
optionally removing protecting groups PG₁ and PG₂.

50. A process for preparing an EGF-like peptide comprising the following sequence:
[SEQ ID NO: 1]
H-Asn¹-Ser²-Asp³-Ser⁴-Glu⁵-Cys⁶-Pro⁷-Leu⁸-Ser⁹-His¹⁰-
Asp¹¹-Gly¹²-Tyr¹³-Cys¹⁴-Leu¹⁵-His¹⁶-Asp¹⁷-Gly¹⁸-Val¹⁹-
Cys²⁰-Met²¹-Tyr²²-Ile²³-Glu²⁴-Ala²⁵-Leu²⁶-Asp²⁷-Lys²⁸-
Tyr²⁹-Ala³⁰-Cys³¹-Asn³²-Cys³³-Val³⁴-Val³⁵-Gly³⁶-Tyr³⁷-
Ile³⁸-Gly³⁹-Glu⁴⁰-Arg⁴¹-Cys⁴²-Gln⁴³-Tyr⁴⁴-Arg⁴⁵-Asp⁴⁶-
Leu⁴⁷-Lys⁴⁸-Trp⁴⁹-Trp⁵⁰-Glu⁵¹-Leu⁵²-Arg⁵³-OH
or a variant thereof,
wherein said process comprises:
coupling a first peptide fragment comprising the sequence:
PG₁-Asn¹-Ser²-Asp³-Ser⁴-Glu⁵-Cys⁶-Pro⁷-Leu⁸-ψSer⁹-His¹⁰-Asp¹¹-Gly¹²-OH [SEQ ID NO: 4] or a variant thereof;
wherein:
PG₁ is an N-terminal protecting group, preferably selected from Boc and Fmoc; and
the C-terminal amino acid is optionally in the form of an activated carboxylic acid derivative;
in solution with a second peptide fragment comprising the sequence:
H-Tyr¹³-Cys¹⁴-Leu¹⁵-His¹⁶-Asp¹⁷-Gly¹⁸-Val¹⁹-Cys²⁰-Met²¹-Tyr²²-Ile²³-Glu²⁴-Ala²⁵-Leu²⁶-AsP²⁷-Lys²⁸-Tyr²⁹-Ala³⁰-Cys³¹-Asn³²-Cys³³-Val³⁴-Val³⁵-Gly³⁶-Tyr³⁷-Ile³⁸-Gly³⁹-Glu⁴⁰-Arg⁴¹-Cys⁴²-Gln⁴³-Tyr⁴⁴-Arg⁴⁵-Asp⁴⁶-Leu⁴⁷-Lys⁴⁸-Trp⁴⁹-Trp⁵⁰-Glu⁵¹-Leu⁵²-Arg⁵³-O-PG₂ [SEQ ID NO: 5] or a variant thereof;
wherein PG₂ is a protecting group, preferably selected from chlorotrityl and t-butyl;
and wherein one or more of the amino acid residues in said first and second peptide fragments is optionally protected, preferably with an acid-cleavable protecting group; and
optionally removing protecting groups PG₁ and PG₂.

51. A process for preparing an EGF-like peptide comprising the following sequence:
H-Val¹-Val²-Ser³-His⁴-Phe⁵-Asn⁶-Asp⁷-Cys⁸-Pro⁹-Asp¹⁰-Ser¹¹-His¹²-Thr¹³-Gln¹⁴-Phe¹⁵-Cys¹⁶-Phe¹⁷-His¹⁸-Gly¹⁹-Thr²⁰-Cys²¹-Arg²²-Phe²³-Leu²⁴-Val²⁵-Gln²⁶-Glu²⁷-Asp²⁸-Lys²⁹-Pro³⁰-Ala³¹-Cys³²-Val³³-Cys³⁴-His³⁵-Ser³⁶-Gly³⁷-Tyr³⁸-Val³⁹-Gly⁴⁰-Ala⁴¹-Arg⁴²-Cys⁴³-Glu⁴⁴-His⁴⁵-Ala⁴⁶-Asp⁴⁷-Leu⁴⁸-Leu⁴⁹-Ala⁵⁰-OH [SEQ ID NO: 6] or a variant thereof;
wherein said process comprises:
coupling a first peptide fragment comprising the sequence:
PG₁-Val¹-Val²-Ser³-His⁴-Phe⁵-Asn⁶-Asp⁷-Cys⁸-Pro⁹-Asp¹⁰-Ser¹¹-His¹²-Thr¹³-Gln¹⁴-Phe¹⁵-Cys¹⁶-Phe¹⁷-His¹⁸-Gly¹⁹-OH [SEQ ID NO: 7] or a variant thereof;
wherein:
PG₁ is an N-terminal protecting group, preferably selected from Boc and Fmoc; and
the C-terminal amino acid is optionally in the form of an activated carboxylic acid derivative;
in solution with a second peptide fragment comprising the sequence:
H-Thr²⁰-Cys²¹-Arg²²-Phe²³-Leu²⁴-Val²⁵-Gln²⁶-Glu²⁷-Asp²⁸-Lys²⁹-Pro³⁰-Ala³¹-Cys³²-Val³³-Cys³⁴-His³⁵-Ser³⁶-Gly³⁷-Tyr³⁸-Val³⁹-Gly⁴⁰-Ala⁴¹-Arg⁴²-Cys⁴³-Glu⁴⁴-His⁴⁵-Ala⁴⁶-Asp⁴⁷-Leu⁴⁸-Leu⁴⁹-Ala⁵⁰-O-PG₂ [SEQ ID NO: 8] or a variant thereof,
wherein PG₂ is a protecting group, preferably selected from chlorotrityl and t-butyl;
and wherein one or more of the amino acid residues in said first and second peptide fragments is optionally protected, preferably with an acid-cleavable protecting group; and
optionally removing protecting groups PG₁ and PG₂.


SEQUENCE LISTING (51 sequences)

SEQ ID NO: 1 - protein, 53 aa, Artificial Sequence
  H-EGF(1-53)-OH
  DISULFID (6)..(6): C6-C20
  DISULFID (14)..(14): C14-C31
  DISULFID (20)..(20): C6-C20
  DISULFID (31)..(31): C14-C31
  DISULFID (33)..(33): C33-C42
  DISULFID (42)..(42): C33-C42
NSDSECPLSHDGYCLHDGVCMYIEALDKYACNCVVGYIGERCQYRDLKWWELR

SEQ ID NO: 2 - protein, 18 aa, Artificial Sequence
  PG1-EGF(1-18)-OH
  MISC_FEATURE (1)..(1): N-terminal protecting group selected from Boc and Fmoc
NSDSECPLSHDGYCLHDG

SEQ ID NO: 3 - protein, 35 aa, Artificial Sequence
  H-EGF(19-53)-OPG2
  MISC_FEATURE (35)..(35): C-terminal protecting group selected from chlorotrityl and t-butyl
VCMYIEALDKYACNCVVGYIGERCQYRDLKWWELR

SEQ ID NO: 4 - protein, 12 aa, Artificial Sequence
  PG1-EGF(1-12)-OH
  MISC_FEATURE (1)..(1): N-terminal protecting group selected from Boc and Fmoc
  MISC_FEATURE (9)..(9): Psi-Ser
NSDSECPLSHDG

SEQ ID NO: 5 - protein, 41 aa, Artificial Sequence
  H-EGF(13-53)-OPG2
  MISC_FEATURE (41)..(41): C-terminal protecting group selected from chlorotrityl and t-butyl
YCLHDGVCMYIEALDKYACNCVVGYIGERCQYRDLKWWELR

SEQ ID NO: 6 - protein, 50 aa, Artificial Sequence
  H-TGF(1-50)-OH
VVSHFNDCPDSHTQFCFHGTCRFLVQEDKPACVCHSGYVGARCEHADLLA

SEQ ID NO: 7 - protein, 19 aa, Artificial Sequence
  PG1-TGF(1-19)-OH
  MISC_FEATURE (1)..(1): N-terminal protecting group selected from Boc and Fmoc
VVSHFNDCPDSHTQFCFHG

SEQ ID NO: 8 - protein, 31 aa, Artificial Sequence
  H-TGF(20-50)-OPG2
  MISC_FEATURE (31)..(31): C-terminal protecting group selected from chlorotrityl and t-butyl
TCRFLVQEDKPACVCHSGYVGARCEHADLLA

SEQ ID NO: 9 - protein, 53 aa, Artificial Sequence; identical to SEQ ID NO: 1
  H-EGF(1-53)-OH
  MISC_FEATURE (1)..(3): Side chain protecting group
  MISC_FEATURE (4)..(4): Psi-Ser
  MISC_FEATURE (5)..(6): Side chain protecting group
  MISC_FEATURE (9)..(9): Psi-Ser
  MISC_FEATURE (10)..(11): Side chain protecting group
  MISC_FEATURE (13)..(14): Side chain protecting group
  MISC_FEATURE (16)..(17): Side chain protecting group
  MISC_FEATURE (20)..(20): Side chain protecting group
  MISC_FEATURE (22)..(22): Side chain protecting group
  MISC_FEATURE (24)..(24): Side chain protecting group
  MISC_FEATURE (27)..(29): Side chain protecting group
  MISC_FEATURE (31)..(33): Side chain protecting group
  MISC_FEATURE (37)..(37): Side chain protecting group
  MISC_FEATURE (40)..(46): Side chain protecting group
  MISC_FEATURE (48)..(51): Side chain protecting group
  MISC_FEATURE (53)..(53): Side chain protecting group

SEQ ID NO: 10 - protein, 18 aa, Artificial Sequence
  PG1-EGF(1-18)-OH
  MISC_FEATURE (1)..(1): N-terminal protecting group selected from Boc and Fmoc
  MISC_FEATURE (1)..(3): side chain protecting group
  MISC_FEATURE (4)..(4): Psi-Ser
  MISC_FEATURE (5)..(6): side chain protecting group
  MISC_FEATURE (9)..(9): Psi-Ser
  MISC_FEATURE (10)..(11): side chain protecting group
  MISC_FEATURE (13)..(14): side chain protecting group
  MISC_FEATURE (16)..(17): side chain protecting group
NSDSECPLSHDGYCLHDG

SEQ ID NO: 11 - protein, 35 aa, Artificial Sequence; identical to SEQ ID NO: 3
  H-EGF(19-53)-OPG2
  MISC_FEATURE (2)..(2): side chain protecting group
  MISC_FEATURE (4)..(4): side chain protecting group
  MISC_FEATURE (6)..(6): side chain protecting group
  MISC_FEATURE (9)..(11): side chain protecting group
  MISC_FEATURE (13)..(15): side chain protecting group
  MISC_FEATURE (19)..(19): side chain protecting group
  MISC_FEATURE (22)..(28): side chain protecting group
  MISC_FEATURE (30)..(33): side chain protecting group
  MISC_FEATURE (35)..(35): side chain protecting group
  MISC_FEATURE (35)..(35): C-terminal protecting group selected from chlorotrityl and t-butyl

SEQ ID NO: 12 - protein, 53 aa, Artificial Sequence; identical to SEQ ID NO: 1
  H-EGF(1-53)-OH
  MISC_FEATURE (1)..(1): Trt side chain protecting group
  MISC_FEATURE (2)..(3): tBu side chain protecting group
  MISC_FEATURE (4)..(4): Psi-Ser
  MISC_FEATURE (5)..(5): tBu side chain protecting group
  MISC_FEATURE (6)..(6): Trt side chain protecting group
  MISC_FEATURE (9)..(9): Psi-Ser
  MISC_FEATURE (10)..(10): Trt side chain protecting group
  MISC_FEATURE (11)..(11): tBu side chain protecting group
  MISC_FEATURE (13)..(13): tBu side chain protecting group
  MISC_FEATURE (14)..(14): Trt side chain protecting group
  MISC_FEATURE (16)..(16): Trt side chain protecting group
  MISC_FEATURE (17)..(17): tBu side chain protecting group
  MISC_FEATURE (20)..(20): Trt side chain protecting group
  MISC_FEATURE (22)..(22): tBu side chain protecting group
  MISC_FEATURE (24)..(24): tBu side chain protecting group
  MISC_FEATURE (27)..(27): tBu side chain protecting group
  MISC_FEATURE (28)..(28): Boc side chain protecting group
  MISC_FEATURE (29)..(29): tBu side chain protecting group
  MISC_FEATURE (31)..(33): Trt side chain protecting group
  MISC_FEATURE (37)..(37): tBu side chain protecting group
  MISC_FEATURE (40)..(40): tBu side chain protecting group
  MISC_FEATURE (41)..(41): Pbf side chain protecting group
  MISC_FEATURE (42)..(43): Trt side chain protecting group
  MISC_FEATURE (44)..(44): tBu side chain protecting group
  MISC_FEATURE (45)..(45): Pbf side chain protecting group
  MISC_FEATURE (46)..(46): tBu side chain protecting group
  MISC_FEATURE (48)..(50): Boc side chain protecting group
  MISC_FEATURE (51)..(51): tBu side chain protecting group
  MISC_FEATURE (53)..(53): Pbf side chain protecting group

SEQ ID NO: 13 - protein, 18 aa, Artificial Sequence
  PG1-EGF(1-18)-OH
  MISC_FEATURE (1)..(1): N-terminal protecting group selected from Boc and Fmoc
  MISC_FEATURE (1)..(1): Trt protecting group
  MISC_FEATURE (2)..(3): tBu protecting group
  MISC_FEATURE (4)..(4): Psi-Ser
  MISC_FEATURE (5)..(5): tBu protecting group
  MISC_FEATURE (6)..(6): Trt protecting group
  MISC_FEATURE (9)..(9): Psi-Ser
  MISC_FEATURE (10)..(10): Trt protecting group
  MISC_FEATURE (11)..(11): tBu protecting group
  MISC_FEATURE (13)..(13): tBu protecting group
  MISC_FEATURE (14)..(14): Trt protecting group
  MISC_FEATURE (16)..(16): Trt protecting group
  MISC_FEATURE (17)..(17): tBu protecting group
NSDSECPLSHDGYCLHDG

SEQ ID NO: 14 - protein, 35 aa, Artificial Sequence; identical to SEQ ID NO: 3
  H-EGF(19-53)-OPG2
  MISC_FEATURE (2)..(2): Trt protecting group
  MISC_FEATURE (4)..(4): tBu protecting group
  MISC_FEATURE (6)..(6): tBu protecting group
  MISC_FEATURE (9)..(9): tBu protecting group
  MISC_FEATURE (10)..(10): Boc protecting group
  MISC_FEATURE (11)..(11): tBu protecting group
  MISC_FEATURE (13)..(15): Trt protecting group
  MISC_FEATURE (19)..(19): tBu protecting group
  MISC_FEATURE (22)..(22): tBu protecting group
  MISC_FEATURE (23)..(23): Pbf protecting group
  MISC_FEATURE (24)..(25): Trt protecting group
  MISC_FEATURE (26)..(26): tBu protecting group
  MISC_FEATURE (27)..(27): Pbf protecting group
  MISC_FEATURE (28)..(28): tBu protecting group
  MISC_FEATURE (30)..(32): Boc protecting group
  MISC_FEATURE (33)..(33): tBu protecting group
  MISC_FEATURE (35)..(35): C-terminal protecting group selected from chlorotrityl and t-butyl
  MISC_FEATURE (35)..(35): Pbf protecting group

SEQ ID NO: 15 - protein, 18 aa, Artificial Sequence
  PG1-EGF(1-18)-OH
  MISC_FEATURE (1)..(1): N-terminal protecting group selected from Boc and Fmoc
  MISC_FEATURE (1)..(3): side chain protecting group
  MISC_FEATURE (4)..(4): Psi-Ser
  MISC_FEATURE (5)..(6): side chain protecting group
  MISC_FEATURE (9)..(9): Psi-Ser
  MISC_FEATURE (10)..(11): side chain protecting group
  MISC_FEATURE (13)..(14): side chain protecting group
  MISC_FEATURE (16)..(17): side chain protecting group
NSDSECPLSHDGYCLHDG

SEQ ID NO: 16 - protein, 12 aa, Artificial Sequence
  PG1-EGF(1-12)-OH
  MISC_FEATURE (1)..(1): N-terminal protecting group selected from Boc and Fmoc
  MISC_FEATURE (1)..(3): side chain protecting group
  MISC_FEATURE (4)..(4): Psi-Ser
  MISC_FEATURE (5)..(6): side chain protecting group
  MISC_FEATURE (9)..(9): Psi-Ser
  MISC_FEATURE (10)..(11): side chain protecting group
NSDSECPLSHDG

SEQ ID NO: 17 - protein, 6 aa, Artificial Sequence
  H-EGF(13-18)-OPG2
  MISC_FEATURE (1)..(2): side chain protecting group
  MISC_FEATURE (4)..(5): side chain protecting group
  MISC_FEATURE (6)..(6): C-terminal protecting group selected from chlorotrityl and t-butyl
YCLHDG

SEQ ID NO: 18 - protein, 12 aa, Artificial Sequence
  PG1-EGF(1-12)-OH
  MISC_FEATURE (1)..(1): N-terminal protecting group selected from Boc and Fmoc
  MISC_FEATURE (1)..(1): Trt protecting group
  MISC_FEATURE (2)..(3): tBu protecting group
  MISC_FEATURE (4)..(4): Psi-Ser
  MISC_FEATURE (5)..(5): tBu protecting group
  MISC_FEATURE (6)..(6): Trt protecting group
  MISC_FEATURE (9)..(9): Psi-Ser
  MISC_FEATURE (10)..(10): Trt protecting group
  MISC_FEATURE (11)..(11): tBu protecting group
NSDSECPLSHDG

SEQ ID NO: 19 - protein, 6 aa, Artificial Sequence
  H-EGF(13-18)-OPG2
  MISC_FEATURE (1)..(1): tBu protecting group
  MISC_FEATURE (2)..(2): Trt protecting group
  MISC_FEATURE (4)..(4): Trt protecting group
  MISC_FEATURE (5)..(5): tBu protecting group
  MISC_FEATURE (6)..(6): C-terminal protecting group selected from chlorotrityl and t-butyl
YCLHDG

SEQ ID NO: 20 - protein, 12 aa, Artificial Sequence
  PG1-EGF(1-12)-OH
  MISC_FEATURE (1)..(1): N-terminal protecting group selected from Boc and Fmoc
  MISC_FEATURE (1)..(3): Side chain protecting group
  MISC_FEATURE (4)..(4): Psi-Ser
  MISC_FEATURE (5)..(6): Side chain protecting group
  MISC_FEATURE (9)..(9): Psi-Ser
  MISC_FEATURE (10)..(11): Side chain protecting group
NSDSECPLSHDG

SEQ ID NO: 21 - protein, 12 aa, Artificial Sequence
  PG1-EGF(1-12)-OH
  MISC_FEATURE (1)..(1): N-terminal protecting group selected from Boc and Fmoc
  MISC_FEATURE (1)..(1): Trt protecting group
  MISC_FEATURE (2)..(3): tBu protecting group
  MISC_FEATURE (4)..(4): Psi-Ser
  MISC_FEATURE (5)..(5): tBu protecting group
  MISC_FEATURE (6)..(6): Trt protecting group
  MISC_FEATURE (9)..(9): Psi-Ser
  MISC_FEATURE (10)..(10): Trt protecting group
  MISC_FEATURE (11)..(11): tBu protecting group
NSDSECPLSHDG

SEQ ID NO: 22 - protein, 18 aa, Artificial Sequence
  PG1-EGF(19-36)-OH
  MISC_FEATURE (1)..(1): N-terminal protecting group selected from Boc and Fmoc
  MISC_FEATURE (2)..(2): side chain protecting group
  MISC_FEATURE (4)..(4): side chain protecting group
  MISC_FEATURE (6)..(6): side chain protecting group
  MISC_FEATURE (9)..(11): side chain protecting group
  MISC_FEATURE (13)..(15): side chain protecting group
VCMYIEALDKYACNCVVG

SEQ ID NO: 23 - protein, 17 aa, Artificial Sequence
  H-EGF(37-53)-OPG2
  MISC_FEATURE (1)..(1): side chain protecting group
  MISC_FEATURE (4)..(10): side chain protecting group
  MISC_FEATURE (12)..(15): side chain protecting group
  MISC_FEATURE (17)..(17): C-terminal protecting group selected from chlorotrityl and t-butyl
  MISC_FEATURE (17)..(17): side chain protecting group
YIGERCQYRDLKWWELR

SEQ ID NO: 24 - protein, 18 aa, Artificial Sequence
  PG1-EGF(19-36)-OH
  MISC_FEATURE (1)..(1): N-terminal protecting group selected from Boc and Fmoc
  MISC_FEATURE (2)..(2): Trt protecting group
  MISC_FEATURE (4)..(4): tBu protecting group
  MISC_FEATURE (6)..(6): tBu protecting group
  MISC_FEATURE (9)..(9): tBu protecting group
  MISC_FEATURE (10)..(10): Boc protecting group
  MISC_FEATURE (11)..(11): tBu protecting group
  MISC_FEATURE (13)..(15): Trt protecting group
VCMYIEALDKYACNCVVG

SEQ ID NO: 25 - protein, 17 aa, Artificial Sequence
  H-EGF(37-53)-OPG2
  MISC_FEATURE (1)..(1): tBu protecting group
  MISC_FEATURE (4)..(4): tBu protecting group
  MISC_FEATURE (5)..(5): Pbf protecting group
  MISC_FEATURE (6)..(7): Trt protecting group
  MISC_FEATURE (8)..(8): tBu protecting group
  MISC_FEATURE (9)..(9): Pbf protecting group
  MISC_FEATURE (10)..(10): tBu protecting group
  MISC_FEATURE (12)..(14): Boc protecting group
  MISC_FEATURE (15)..(15): tBu protecting group
  MISC_FEATURE (17)..(17): C-terminal protecting group selected from chlorotrityl and t-butyl
  MISC_FEATURE (17)..(17): Pbf protecting group
YIGERCQYRDLKWWELR

SEQ ID NO: 26 - protein, 21 aa, Artificial Sequence
  PG1-EGF(19-36)-OH
  MISC_FEATURE (1)..(1): N-terminal protecting group selected from Boc and Fmoc
  MISC_FEATURE (2)..(2): side chain protecting group
  MISC_FEATURE (4)..(4): side chain protecting group
  MISC_FEATURE (6)..(6): side chain protecting group
  MISC_FEATURE (9)..(11): side chain protecting group
  MISC_FEATURE (13)..(15): side chain protecting group
  MISC_FEATURE (19)..(19): side chain protecting group
VCMYIEALDKYACNCVVGYIG

SEQ ID NO: 27 - protein, 14 aa, Artificial Sequence
  H-EGF(40-53)-OPG2
  MISC_FEATURE (1)..(7): side chain protecting group
  MISC_FEATURE (9)..(12): side chain protecting group
  MISC_FEATURE (14)..(14): side chain protecting group
  MISC_FEATURE (14)..(14): C-terminal protecting group selected from chlorotrityl and t-butyl
ERCQYRDLKWWELR

SEQ ID NO: 28 - protein, 21 aa, Artificial Sequence
  PG1-EGF(19-36)-OH
  MISC_FEATURE (1)..(1): N-terminal protecting group selected from Boc and Fmoc
  MISC_FEATURE (2)..(2): Trt protecting group
  MISC_FEATURE (4)..(4): tBu protecting group
  MISC_FEATURE (6)..(6): tBu protecting group
  MISC_FEATURE (9)..(9): tBu protecting group
  MISC_FEATURE (10)..(10): Boc protecting group
  MISC_FEATURE (11)..(11): tBu protecting group
  MISC_FEATURE (13)..(15): Trt protecting group
  MISC_FEATURE (19)..(19): tBu protecting group
  MISC_FEATURE (19)..(19): side chain protecting group
VCMYIEALDKYACNCVVGYIG

SEQ ID NO: 29 - protein, 14 aa, Artificial Sequence
  H-EGF(40-53)-OPG2
  MISC_FEATURE (1)..(1): tBu protecting group
  MISC_FEATURE (2)..(2): Pbf protecting group
  MISC_FEATURE (3)..(4): Trt protecting group
  MISC_FEATURE (5)..(5): tBu protecting group
  MISC_FEATURE (6)..(6): Pbf protecting group
  MISC_FEATURE (7)..(7): tBu protecting group
  MISC_FEATURE (9)..(11): Boc protecting group
  MISC_FEATURE (12)..(12): tBu protecting group
  MISC_FEATURE (14)..(14): Pbf protecting group
  MISC_FEATURE (14)..(14): C-terminal protecting group selected from chlorotrityl and t-butyl
ERCQYRDLKWWELR

SEQ ID NO: 30 - protein, 3 aa, Artificial Sequence
  PG1-EGF(37-39)-OH
  MISC_FEATURE (1)..(1): N-terminal protecting group selected from Boc and Fmoc
  MISC_FEATURE (1)..(1): side chain protecting group
YIG

SEQ ID NO: 31 - protein, 3 aa, Artificial Sequence
  PG1-EGF(37-39)-OH
  MISC_FEATURE (1)..(1): N-terminal protecting group selected from Boc and Fmoc
  MISC_FEATURE (1)..(1): tBu protecting group
YIG

SEQ ID NO: 32 - protein, 41 aa, Artificial Sequence; identical to SEQ ID NO: 5
  H-EGF(13-53)-OPG2
  MISC_FEATURE (1)..(2): protecting side group
  MISC_FEATURE (4)..(5): protecting side group
  MISC_FEATURE (8)..(8): protecting side group
  MISC_FEATURE (10)..(10): protecting side group
  MISC_FEATURE (12)..(12): protecting side group
  MISC_FEATURE (15)..(17): protecting side group
  MISC_FEATURE (19)..(21): protecting side group
  MISC_FEATURE (25)..(25): protecting side group
  MISC_FEATURE (28)..(34): protecting side group
  MISC_FEATURE (36)..(39): protecting side group
  MISC_FEATURE (41)..(41): C-terminal protecting group selected from chlorotrityl and t-butyl
  MISC_FEATURE (41)..(41): protecting side group

SEQ ID NO: 33 - protein, 41 aa, Artificial Sequence; identical to SEQ ID NO: 5
  H-EGF(13-53)-OPG2
  MISC_FEATURE (1)..(1): tBu protecting side group
  MISC_FEATURE (2)..(2): Trt protecting side group
  MISC_FEATURE (4)..(4): Trt protecting side group
  MISC_FEATURE (5)..(5): tBu protecting side group
  MISC_FEATURE (8)..(8): Trt protecting side group
  MISC_FEATURE (10)..(10): tBu protecting side group
  MISC_FEATURE (12)..(12): tBu protecting side group
  MISC_FEATURE (15)..(15): tBu protecting side group
  MISC_FEATURE (16)..(16): Boc protecting side group
  MISC_FEATURE (17)..(17): tBu protecting side group
  MISC_FEATURE (19)..(21): Trt protecting side group
  MISC_FEATURE (25)..(25): tBu protecting side group
  MISC_FEATURE (28)..(28): tBu protecting side group
  MISC_FEATURE (29)..(29): Pbf protecting side group
  MISC_FEATURE (30)..(31): Trt protecting side group
  MISC_FEATURE (32)..(32): tBu protecting side group
  MISC_FEATURE (33)..(33): Pbf protecting side group
  MISC_FEATURE (34)..(34): tBu protecting side group
  MISC_FEATURE (36)..(38): Boc protecting side group
  MISC_FEATURE (39)..(39): tBu protecting side group
  MISC_FEATURE (41)..(41): C-terminal protecting group selected from chlorotrityl and t-butyl
  MISC_FEATURE (41)..(41): Pbf protecting side group

SEQ ID NO: 34 - protein, 12 aa, Artificial Sequence
  PG1-EGF(1-12)-OH
  MISC_FEATURE (1)..(1): N-terminal protecting group selected from Boc and Fmoc
  MISC_FEATURE (1)..(3): Side chain protecting group
  MISC_FEATURE (4)..(4): Psi-Ser
  MISC_FEATURE (5)..(6): Side chain protecting group
  MISC_FEATURE (9)..(9): Psi-Ser
  MISC_FEATURE (10)..(11): Side chain protecting group
NSDSECPLSHDG

SEQ ID NO: 35 - protein, 24 aa, Artificial Sequence
  PG1-EGF(13-36)-OH
  MISC_FEATURE (1)..(1): N-terminal protecting group selected from Boc or Fmoc
  MISC_FEATURE (1)..(2): protecting side chain
  MISC_FEATURE (4)..(5): protecting side chain
  MISC_FEATURE (8)..(8): protecting side chain
  MISC_FEATURE (10)..(10): protecting side chain
  MISC_FEATURE (12)..(12): protecting side chain
  MISC_FEATURE (15)..(17): protecting side chain
  MISC_FEATURE (19)..(21): protecting side chain
YCLHDGVCMYIEALDKYACNCVVG

SEQ ID NO: 36 - protein, 24 aa, Artificial Sequence
  PG1-EGF(13-36)-OH
  MISC_FEATURE (1)..(1): N-terminal protecting group selected from Boc or Fmoc
  MISC_FEATURE (1)..(1): tBu side chain
  MISC_FEATURE (2)..(2): Trt side chain
  MISC_FEATURE (4)..(4): Trt side chain
  MISC_FEATURE (5)..(5): tBu side chain
  MISC_FEATURE (8)..(8): Trt side chain
  MISC_FEATURE (10)..(10): tBu side chain
  MISC_FEATURE (12)..(12): tBu side chain
  MISC_FEATURE (15)..(15): tBu side chain
  MISC_FEATURE (16)..(16): Boc side chain
  MISC_FEATURE (17)..(17): tBu side chain
  MISC_FEATURE (19)..(21): Trt side chain
YCLHDGVCMYIEALDKYACNCVVG

SEQ ID NO: 37 - protein, 50 aa, Artificial Sequence; identical to SEQ ID NO: 6
  PG1-TGF(1-50)-OPG2
  MISC_FEATURE (1)..(1): N-terminal protecting group selected from Boc or Fmoc
  MISC_FEATURE (3)..(3): side chain protecting group
  MISC_FEATURE (4)..(4): side chain protecting group
  MISC_FEATURE (6)..(6): side chain protecting group
  MISC_FEATURE (7)..(7): side chain protecting group
  MISC_FEATURE (8)..(8): side chain protecting group
  MISC_FEATURE (10)..(10): side chain protecting group
  MISC_FEATURE (11)..(11): Psi-Ser
  MISC_FEATURE (12)..(12): side chain protecting group
  MISC_FEATURE (13)..(13): side chain protecting group
  MISC_FEATURE (14)..(14): side chain protecting group
  MISC_FEATURE (16)..(16): side chain protecting group
  MISC_FEATURE (18)..(18): side chain protecting group
  MISC_FEATURE (20)..(20): side chain protecting group
  MISC_FEATURE (21)..(21): side chain protecting group
  MISC_FEATURE (22)..(22): side chain protecting group
  MISC_FEATURE (27)..(27): side chain protecting group
  MISC_FEATURE (28)..(28): side chain protecting group
  MISC_FEATURE (29)..(29): side chain protecting group
  MISC_FEATURE (32)..(32): side chain protecting group
  MISC_FEATURE (34)..(34): side chain protecting group
  MISC_FEATURE (36)..(36): side chain protecting group
  MISC_FEATURE (38)..(38): side chain protecting group
  MISC_FEATURE (42)..(42): side chain protecting group
  MISC_FEATURE (43)..(43): side chain protecting group
  MISC_FEATURE (44)..(44): side chain protecting group
  MISC_FEATURE (45)..(45): side chain protecting group
  MISC_FEATURE (47)..(47): side chain protecting group
  MISC_FEATURE (50)..(50): C-terminal protecting group selected from chlorotrityl and t-butyl

SEQ ID NO: 38 - protein, 19 aa, Artificial Sequence
  PG1-TGF(1-19)-OH
  MISC_FEATURE (1)..(1): N-terminal protecting group selected from Boc or Fmoc
  MISC_FEATURE (3)..(3): side chain protecting group
  MISC_FEATURE (4)..(4): side chain protecting group
  MISC_FEATURE (6)..(6): side chain protecting group
  MISC_FEATURE (7)..(7): side chain protecting group
  MISC_FEATURE (8)..(8): side chain protecting group
  MISC_FEATURE (10)..(10): side chain protecting group
  MISC_FEATURE (11)..(11): Psi-Ser
  MISC_FEATURE (12)..(12): side chain protecting group
  MISC_FEATURE (13)..(13): side chain protecting group
  MISC_FEATURE (14)..(14): side chain protecting group
  MISC_FEATURE (16)..(16): side chain protecting group
  MISC_FEATURE (18)..(18): side chain protecting group
VVSHFNDCPDSHTQFCFHG

SEQ ID NO: 39 - protein, 31 aa, Artificial Sequence; identical to SEQ ID NO: 8
  H-TGF(20-50)-OPG2
  MISC_FEATURE (1)..(1): side chain protecting group
  MISC_FEATURE (2)..(2): side chain protecting group
  MISC_FEATURE (3)..(3): side chain protecting group
  MISC_FEATURE (8)..(8): side chain protecting group
  MISC_FEATURE (9)..(9): side chain protecting group
  MISC_FEATURE (10)..(10): side chain protecting group
  MISC_FEATURE (13)..(13): side chain protecting group
  MISC_FEATURE (15)..(15): side chain protecting group
  MISC_FEATURE (17)..(17): side chain protecting group
  MISC_FEATURE (19)..(19): side chain protecting group
  MISC_FEATURE (23)..(23): side chain protecting group
  MISC_FEATURE (24)..(24): side chain protecting group
  MISC_FEATURE (25)..(25): side chain protecting group
  MISC_FEATURE (26)..(26): side chain protecting group
  MISC_FEATURE (28)..(28): side chain protecting group
  MISC_FEATURE (31)..(31): C-terminal protecting group selected from chlorotrityl and t-butyl

SEQ ID NO: 40 - protein, 50 aa, Artificial Sequence; identical to SEQ ID NO: 6
  PG1-TGF(1-50)-OPG2
  MISC_FEATURE (1)..(1): N-terminal protecting group selected from Boc or Fmoc
  MISC_FEATURE (3)..(3): tBu protecting group
  MISC_FEATURE (4)..(4): Trt protecting group
  MISC_FEATURE (6)..(6): Trt protecting group
  MISC_FEATURE (7)..(7): tBu protecting group
  MISC_FEATURE (8)..(8): Trt protecting group
  MISC_FEATURE (10)..(10): tBu protecting group
  MISC_FEATURE (11)..(11): Psi-Ser
  MISC_FEATURE (12)..(12): Trt protecting group
  MISC_FEATURE (13)..(13): tBu protecting group
  MISC_FEATURE (14)..(14): Trt protecting group
  MISC_FEATURE (16)..(16): Trt protecting group
  MISC_FEATURE (18)..(18): Trt protecting group
  MISC_FEATURE (20)..(20): tBu protecting group
  MISC_FEATURE (21)..(21): Trt protecting group
  MISC_FEATURE (22)..(22): Pbf protecting group
  MISC_FEATURE (27)..(27): tBu protecting group
  MISC_FEATURE (28)..(28): tBu protecting group
  MISC_FEATURE (29)..(29): Boc protecting group
  MISC_FEATURE (32)..(32): Trt protecting group
  MISC_FEATURE (34)..(34): Trt protecting group
  MISC_FEATURE (36)..(36): tBu protecting group
  MISC_FEATURE (38)..(38): tBu protecting group
  MISC_FEATURE (42)..(42): Pbf protecting group
  MISC_FEATURE (43)..(43): Trt protecting group
  MISC_FEATURE (44)..(44): tBu protecting group
  MISC_FEATURE (45)..(45): Trt protecting group
  MISC_FEATURE (47)..(47): tBu protecting group
  MISC_FEATURE (50)..(50): C-terminal protecting group selected from chlorotrityl and t-butyl

SEQ ID NO: 41 - protein, 19 aa, Artificial Sequence
  PG1-TGF(1-19)-OH
  MISC_FEATURE (1)..(1): N-terminal protecting group selected from Boc or Fmoc
  MISC_FEATURE (3)..(3): tBu protecting group
  MISC_FEATURE (4)..(4): Trt protecting group
  MISC_FEATURE (6)..(6): Trt protecting group
  MISC_FEATURE (7)..(7): tBu protecting group
  MISC_FEATURE (8)..(8): Trt protecting group
  MISC_FEATURE (10)..(10): tBu protecting group
  MISC_FEATURE (11)..(11): Psi-Ser
  MISC_FEATURE (12)..(12): Trt protecting group
  MISC_FEATURE (13)..(13): tBu protecting group
  MISC_FEATURE (14)..(14): Trt protecting group
  MISC_FEATURE (16)..(16): Trt protecting group
  MISC_FEATURE (18)..(18): Trt protecting group
VVSHFNDCPDSHTQFCFHG

SEQ ID NO: 42 - protein, 31 aa, Artificial Sequence; identical to SEQ ID NO: 8
  H-TGF(20-50)-OPG2
  MISC_FEATURE (1)..(1): tBu protecting group
  MISC_FEATURE (2)..(2): Trt protecting group
  MISC_FEATURE (3)..(3): Pbf protecting group
  MISC_FEATURE (8)..(8): tBu protecting group
  MISC_FEATURE (9)..(9): tBu protecting group
  MISC_FEATURE (10)..(10): Boc protecting group
  MISC_FEATURE (13)..(13): Trt protecting group
  MISC_FEATURE (15)..(15): Trt protecting group
  MISC_FEATURE (17)..(17): tBu protecting group
  MISC_FEATURE (19)..(19): tBu protecting group
  MISC_FEATURE (23)..(23): Pbf protecting group
  MISC_FEATURE (24)..(24): Trt protecting group
  MISC_FEATURE (25)..(25): tBu protecting group
  MISC_FEATURE (26)..(26): Trt protecting group
  MISC_FEATURE (28)..(28): tBu protecting group
  MISC_FEATURE (31)..(31): C-terminal protecting group selected from chlorotrityl and t-butyl

SEQ ID NO: 43 - protein, 18 aa, Artificial Sequence
  PG1-TGF(20-37)-OH
  MISC_FEATURE (1)..(1): N-terminal protecting group selected from Boc and Fmoc
  MISC_FEATURE (1)..(1): side chain protecting group
  MISC_FEATURE (2)..(2): side chain protecting group
  MISC_FEATURE (3)..(3): side chain protecting group
  MISC_FEATURE (8)..(8): side chain protecting group
  MISC_FEATURE (9)..(9): side chain protecting group
  MISC_FEATURE (10)..(10): side chain protecting group
  MISC_FEATURE (13)..(13): side chain protecting group
  MISC_FEATURE (15)..(15): side chain protecting group
  MISC_FEATURE (17)..(17): side chain protecting group
TCRFLVQEDKPACVCHSG

SEQ ID NO: 44 - protein, 13 aa, Artificial Sequence
  H-TGF(38-50)-OPG2
  MISC_FEATURE (1)..(1): side chain protecting group
  MISC_FEATURE (5)..(5): side chain protecting group
  MISC_FEATURE (6)..(6): side chain protecting group
  MISC_FEATURE (7)..(7): side chain protecting group
  MISC_FEATURE (8)..(8): side chain protecting group
  MISC_FEATURE (10)..(10): side chain protecting group
  MISC_FEATURE (13)..(13): C-terminal protecting group selected from chlorotrityl and t-butyl
YVGARCEHADLLA

SEQ ID NO: 45 - protein, 31 aa, Artificial Sequence; identical to SEQ ID NO: 8
  H-TGF(20-50)-OPG2
  MISC_FEATURE (1)..(1): tBu protecting group
  MISC_FEATURE (2)..(2): Trt protecting group
  MISC_FEATURE (3)..(3): Pbf protecting group
  MISC_FEATURE (8)..(8): tBu protecting group
  MISC_FEATURE (9)..(9): tBu protecting group
  MISC_FEATURE (10)..(10): Boc protecting group
  MISC_FEATURE (13)..(13): Trt protecting group
  MISC_FEATURE (15)..(15): Trt protecting group
  MISC_FEATURE (17)..(17): tBu protecting group
  MISC_FEATURE (19)..(19): tBu protecting group
  MISC_FEATURE (23)..(23): Pbf protecting group
  MISC_FEATURE (24)..(24): Trt protecting group
  MISC_FEATURE (25)..(25): tBu protecting group
  MISC_FEATURE (26)..(26): Trt protecting group
  MISC_FEATURE (28)..(28): tBu protecting group
  MISC_FEATURE (31)..(31): C-terminal protecting group selected from chlorotrityl and t-butyl

SEQ ID NO: 46 - protein, 18 aa, Artificial Sequence
  PG1-TGF(20-37)-OH
  MISC_FEATURE (1)..(1): N-terminal protecting group selected from Boc and Fmoc
  MISC_FEATURE (1)..(1): tBu protecting group
  MISC_FEATURE (2)..(2): Trt protecting group
  MISC_FEATURE (3)..(3): Pbf protecting group
  MISC_FEATURE (8)..(8): tBu protecting group
  MISC_FEATURE (9)..(9): tBu protecting group
  MISC_FEATURE (10)..(10): Boc protecting group
  MISC_FEATURE (13)..(13): Trt protecting group
  MISC_FEATURE (15)..(15): Trt protecting group
  MISC_FEATURE (17)..(17): tBu protecting group
TCRFLVQEDKPACVCHSG

SEQ ID NO: 47 - protein, 13 aa, Artificial Sequence
  H-TGF(38-50)-OPG2
  MISC_FEATURE (1)..(1): tBu protecting group
  MISC_FEATURE (5)..(5): Pbf protecting group
  MISC_FEATURE (6)..(6): Trt protecting group
  MISC_FEATURE (7)..(7): tBu protecting group
  MISC_FEATURE (8)..(8): Trt protecting group
  MISC_FEATURE (10)..(10): tBu protecting group
  MISC_FEATURE (13)..(13): C-terminal protecting group selected from chlorotrityl and t-butyl
YVGARCEHADLLA

SEQ ID NO: 48 - protein, 13 aa, Artificial Sequence
  H-TGF(38-50)-OPG2
  MISC_FEATURE (1)..(1): side chain protecting group
  MISC_FEATURE (5)..(5): side chain protecting group
  MISC_FEATURE (6)..(6): side chain protecting group
  MISC_FEATURE (7)..(7): side chain protecting group
  MISC_FEATURE (8)..(8): side chain protecting group
  MISC_FEATURE (10)..(10): side chain protecting group
  MISC_FEATURE (13)..(13): C-terminal protecting group selected from chlorotrityl and t-butyl
YVGARCEHADLLA

SEQ ID NO: 49 - protein, 11 aa, Artificial Sequence
  Generic
  DISULFID (1)..(1): C1-C3
  MISC_FEATURE (2)..(2): (X)7, where each X is independently a natural or unnatural amino acid
  DISULFID (3)..(3): C2-C4
  MISC_FEATURE (4)..(4): (X)4-5, where each X is independently a natural or unnatural amino acid
  DISULFID (5)..(5): C1-C3
  MISC_FEATURE (6)..(6): (X)10-13, where each X is independently a natural or unnatural amino acid
  DISULFID (7)..(7): C2-C4
  MISC_FEATURE (8)..(8): (X), where each X is independently a natural or unnatural amino acid
  DISULFID (9)..(9): C5-C6
  MISC_FEATURE (10)..(10): (X)8, where each X is independently a natural or unnatural amino acid
  DISULFID (11)..(11): C5-C6
CXCXCXCXCXC

SEQ ID NO: 50 - protein, 50 aa, Artificial Sequence; identical to SEQ ID NO: 6
  H-TGF(1-50)-OH
  MISC_FEATURE (3)..(3): side chain protecting group
  MISC_FEATURE (4)..(4): side chain protecting group
  MISC_FEATURE (6)..(6): side chain protecting group
  MISC_FEATURE (7)..(7): side chain protecting group
  MISC_FEATURE (8)..(8): side chain protecting group
  MISC_FEATURE (10)..(10): side chain protecting group
  MISC_FEATURE (11)..(11): Psi-Ser
  MISC_FEATURE (12)..(12): side chain protecting group
  MISC_FEATURE (13)..(13): side chain protecting group
  MISC_FEATURE (14)..(14): side chain protecting group
  MISC_FEATURE (16)..(16): side chain protecting group
  MISC_FEATURE (18)..(18): side chain protecting group
  MISC_FEATURE (20)..(20): side chain protecting group
  MISC_FEATURE (21)..(21): side chain protecting group
  MISC_FEATURE (22)..(22): side chain protecting group
  MISC_FEATURE (27)..(27): side chain protecting group
  MISC_FEATURE (28)..(28): side chain protecting group
  MISC_FEATURE (29)..(29): side chain protecting group
  MISC_FEATURE (32)..(32): side chain protecting group
  MISC_FEATURE (34)..(34): side chain protecting group
  MISC_FEATURE (36)..(36): side chain protecting group
  MISC_FEATURE (38)..(38): side chain protecting group
  MISC_FEATURE (42)..(42): side chain protecting group
  MISC_FEATURE (43)..(43): side chain protecting group
  MISC_FEATURE (44)..(44): side chain protecting group
  MISC_FEATURE (45)..(45): side chain protecting group
  MISC_FEATURE (47)..(47): side chain protecting group

SEQ ID NO: 51 - protein, 50 aa, Artificial Sequence; identical to SEQ ID NO: 6
  H-TGF(1-50)-OH
  MISC_FEATURE (3)..(3): tBu protecting group
  MISC_FEATURE (4)..(4): Trt protecting group
  MISC_FEATURE (6)..(6): Trt protecting group
  MISC_FEATURE (7)..(7): tBu protecting group
  MISC_FEATURE (8)..(8): Trt protecting group
  MISC_FEATURE (10)..(10): tBu protecting group
  MISC_FEATURE (11)..(11): Psi-Ser
  MISC_FEATURE (12)..(12): Trt protecting group
  MISC_FEATURE (13)..(13): tBu protecting group
  MISC_FEATURE (14)..(14): Trt protecting group
  MISC_FEATURE (16)..(16): Trt protecting group
  MISC_FEATURE (18)..(18): Trt protecting group
  MISC_FEATURE (20)..(20): tBu protecting group
  MISC_FEATURE (21)..(21): Trt protecting group
  MISC_FEATURE (22)..(22): Pbf protecting group
  MISC_FEATURE (27)..(27): tBu protecting group
  MISC_FEATURE (28)..(28): tBu protecting group
  MISC_FEATURE (29)..(29): Boc protecting group
  MISC_FEATURE (32)..(32): Trt protecting group
  MISC_FEATURE (34)..(34): Trt protecting group
  MISC_FEATURE (36)..(36): tBu protecting group
  MISC_FEATURE (38)..(38): tBu protecting group
  MISC_FEATURE (42)..(42): Pbf protecting group
  MISC_FEATURE (43)..(43): Trt protecting group
  MISC_FEATURE (44)..(44): tBu protecting group
  MISC_FEATURE (45)..(45): Trt protecting group
  MISC_FEATURE (47)..(47): tBu protecting group